(19) United States
(12) Patent Application Publication
(10) Pub. No.: US 2010/0298221 A1
(43) Pub. Date: Nov. 25, 2010

(54) 2-PHENOXY NICOTINE ACID DERIVATIVE AND USE THEREOF

(75) Inventors: Heinrich Meier, Wuppertal (DE); Peter kolkhof, Wuppertal (DE); Axel Kretschmer, Wuppertal (GB); Arounarith Tuch, Frankreich (FR); Lars Barfacker, Oberhausen (DE); Yolanda Cancho Grande, Leverkusen (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/440,725

(22) PCT Filed: Aug. 30, 2007
(86) PCT No.: PCT/EP2007/007575

§ 371 Date: Nov. 24, 2009

(30) Foreign Application Priority Data
Sep. 12, 2006 (DE) 10 2006 043 520.6

(51) Int. Cl. A61K 38/16; C07D 213/80; C07D 213/22; A61K 31/444; A61K 31/4412; A61K 31/546; A61K 31/585; A61K 31/55; A61K 31/551; A61K 31/60; A61K 31/4365; A61K 31/519; A61P 3/00; A61P 9/10; A61P 9/00

(52) U.S. Cl. 514/13.5; 546/298; 546/258; 514/334; 514/350; 514/222.5; 514/173; 514/215; 514/213.01; 514/220; 514/165; 514/301; 514/262.1

(57) ABSTRACT

The present invention relates to novel 2-phenoxy-6-phenyl- and 2-phenoxy-6-pyridylnicotinic acid derivatives, to processes for their preparation, to their use for the treatment and/or prophylaxis of diseases and to their use for producing medicaments for the treatment and/or prophylaxis of diseases, preferably for the treatment and/or prophylaxis of cardiovascular disorders, especially of dyslipidemias, arteriosclerosis and heart failure.

The present application relates to novel 2-phenoxy-6-phenyl- and 2-phenoxy-6-pyridylnicotinic acid derivatives, to processes for their preparation, to their use for the treatment and/or prophylaxis of diseases and to their use for producing medicaments for the treatment and/or prophylaxis of diseases, preferably for the treatment and/or prophylaxis of cardiovascular disorders, especially of dyslipidemias, arteriosclerosis and heart failure.

In spite of many therapeutic successes, cardiovascular disorders remain a serious public health problem. While treatment with statins by inhibiting HMG-CoA reductase very successfully lower both the plasma concentration of LDL cholesterol (LDL-C) and the mortality of patients at risk, there is currently a lack of convincing treatment strategies for the therapy of patients with unfavorable HDL-C/LDL-C ratio or with hypertriglyceridemia.

Apart from niacin, fibrates are to date the only therapy option for patients of these risk groups. They lower elevated triglycerides by 20-50%, lower LDL-C by 10-15%, alter the LDL particle size of atherogenic low-density LDL to normal-density and less dense atherogenic LDL and increase the HDL concentrations by 10-15%.

Fibrates act as weak agonsists of the peroxisome proliferator-activated receptor (PPAR)-alpha (Nature 1990, 347, 645-50). PPAR-alpha is a nuclear receptor which regulates the expression of target genes by binding to DNA sequences in the promoter region of these genes [also known as PPAR Response Elements (PPREs)]. PPREs have been identified in a series of genes which code for proteins which regulate lipid metabolism. PPAR-alpha is expressed to a high degree in the liver and its activation leads to effects including lowered VLDL production/secretion and reduced apolipoprotein CIII (ApoCIII) synthesis. In contrast, the synthesis of apolipoprotein A1 (ApoA1) is enhanced.

One disadvantage of fibrates approved to date is their only weak interaction with the receptor (EC₅₀ in the μM range), which leads in turn to the above-described relatively minor pharmacological effects.

It was an object of the present invention to provide novel compounds which can be used as PPAR-alpha modulators for the treatment and/or prophylaxis especially of cardiovascular disorders.

WO 98/45268 claims nicotinamide derivatives with PDE 4D- and TNF-inhibitory activity for the treatment of respiratory pathway disorders and allergic, inflammatory and rheumatoid disorders. WO 02/30358 claims various heteroaromatic compounds as modulators of the CCR4 chemokine receptor function for the treatment of allergic disorders. Variously substituted 2-arylpyridines are disclosed in US 2003/0152520 as CRF receptor modulators for the treatment of states of anxiety and depression. US 2006/0063779 describes substituted pyridine derivatives and their use for the treatment of cancers. WO 2006/097220 claims 4-phenoxy-2-phenylpyrimidinecarboxylic acids as PPAR-alpha modulators for the treatment of dyslipidemias and arteriosclerosis.

The present invention provides compounds of the general formula (I)

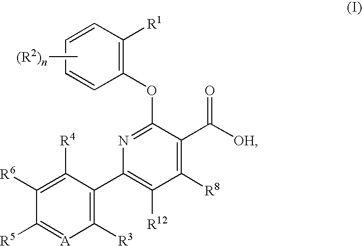

in which
R¹ is halogen, cyano, (C₁-C₄)-alkyl, trifluoromethyl, (C₁-C₄)-alkoxy or trifluoromethoxy,
R² is a substituent selected from the group of halogen, cyano, (C₁-C₆)-alkyl, (C₁-C₆)-alkoxy and —NR⁹—C(═O)—R¹⁰, in which alkyl and alkoxy may in turn be substituted by hydroxyl, (C₁-C₄)-alkoxy, amino, mono-(C₁-C₄)-alkylamino or di-(C₁-C₄)-alkylamino, or up to pentasubstituted by fluorine, and
   R⁹ is hydrogen or (C₁-C₆)-alkyl
   and
   R¹⁰ is hydrogen, (C₁-C₆)-alkyl or (C₁-C₆)-alkoxy,
n is 0, 1, 2 or 3,
   where, in the case that the substituent R² occurs more than once, its definitions may be identical or different,
A is N or C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to pentasubstituted by fluorine,
R⁸ is hydrogen, methyl or trifluoromethyl
   and
R¹² is hydrogen or fluorine,
   and the salts, solvates and solvates of the salts thereof.

Inventive compounds are the compounds of the formula (I) and the salts, solvates and solvates of the salts thereof, the compounds, encompassed by formula (I), of the formulae mentioned below and the salts, solvates and solvates of the salts thereof, and also the compounds which are encompassed by the formula (I) and are cited below as working examples and the salts, solvates and solvates of the salts thereof if the compounds which are encompassed by the formula (I) and are cited below are not already salts, solvates and solvates of the salts.

Depending on their structure, the inventive compounds can exist in stereoisomeric forms (enantiomers, diastereomers). Accordingly, the invention encompasses the enantiomers or diastereomers and their particular mixtures. From such mixtures of enantiomers and/or diastereomers, it is possible to isolate the stereoisomerically uniform components in a known manner.

If the inventive compounds can occur in tautomeric forms, the present invention encompasses all tautomeric forms.

In the context of the present invention, preferred salts are physiologically acceptable salts of the inventive compounds. The invention also comprises salts which themselves are unsuitable for pharmaceutical applications, but which can be used, for example, for isolating or purifying the inventive compounds.

Physiologically acceptable salts of the inventive compounds include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of customary bases, such as, by way of example and with preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and with preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the inventive compounds which, in the solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the inventive compounds. The term “prodrugs” includes compounds which may themselves be biologically active or inactive but which, during their time of residence in the body, are converted into inventive compounds (for example metabolically or hydrolytically).

In particular, the present invention also encompasses hydrolyzable ester derivatives of the carboxylic acids of the formula (I). This is understood to mean esters which can be hydrolyzed to the free carboxylic acids in physiological media and especially in vivo by an enzymatic or chemical route. Preferred esters of this kind are straight-chain or branched (C₁-C₆)-alkyl esters in which the alkyl group may be substituted by hydroxyl, (C₁-C₄)-alkoxy, amino, mono-(C₁-C₄)-alkylamino and/or di-(C₁-C₄)-alkylamino. Particular preference is given to the methyl or ethyl esters of the compounds of the formula (I).

In the context of the present invention, unless specified otherwise, the substituents are each defined as follows:

In the context of the invention, (C₁-C₆)-alkyl and (C₁-C₄)-alkyl are each a straight-chain or branched alkyl radical having from 1 to 6 and from 1 to 4 carbon atoms respectively. Preference is given to a straight-chain or branched alkyl radical having from 1 to 4 carbon atoms. Preferred examples include: methyl, ethyl, n-propyl, isopropyl, n-butyl, isobutyl, sec-butyl, tert-butyl, 1-ethylpropyl, n-pentyl, isopentyl and n-hexyl.

In the context of the invention, (C₁-C₆)-alkoxy and (C₁-C₄)-alkoxy are each a straight-chain or branched alkoxy radical having from 1 to 6 and from 1 to 4 carbon atoms respectively. Preference is given to a straight-chain or branched alkoxy radical having from 1 to 4 carbon atoms. Preferred examples include: methoxy, ethoxy, n-propoxy, isopropoxy, n-butoxy, tert-butoxy, n-pentoxy and n-hexoxy.

In the context of the invention, mono-(C₁-C₄)-alkylamino is an amino group having a straight-chain or branched alkyl substituent having from 1 to 4 carbon atoms. Preferred examples include: methyl-amino, ethylamino, n-propylamino, isopropylamino, n-butylamino and tert-butylamino.

In the context of the invention, di-(C₁-C₄)-alkylamino is an amino group having two identical or different straight-chain or branched alkyl substituents which each have from 1 to 4 carbon atoms. Preferred examples include: N,N-dimethylamino, N,N-diethylamino, N-ethyl-N-methylamino, N-methyl-N-n-propylamino, N-isopropyl-N-n-methylamino, N,N-diisopropylamino, N-n-butyl-N-methylamino and N-tert-butyl-N-methylamino.

In the context of the invention, halogen includes fluorine, chlorine, bromine and iodine. Preference is given to chlorine or fluorine.

When radicals in the inventive compounds are substituted, the radicals may, unless specified otherwise, be mono- or polysubstituted. In the context of the present invention, the definitions of radicals which occur more than once are independent of one another. Substitution with one, two or three identical or different substituents is preferred. Very particular preference is given to substitution by one substituent.

In the context of the present invention, preference is given to compounds of the formula (I) in which
R¹ is halogen, cyano or (C₁-C₄)-alkyl,
R² is a substituent selected from the group of halogen, cyano, (C₁-C₆)-alkoxy and —NR⁹—C(═O)—R¹⁰, in which alkyl and alkoxy may in turn be substituted by hydroxyl, (C₁-C₄)-alkoxy, amino, mono-(C₁-C₄)-alkylamino or di-(C₁-C₄)-alkylamino, or up to pentasubstituted by fluorine, and
   R⁹ is hydrogen or (C₁-C₆)-alkyl
   and
   R¹⁰ is hydrogen, (C₁-C₆)-alkyl or (C₁-C₆)-alkoxy,
n is 0, 1, 2 or 3,
   where, in the case that the substituent R² occurs more than once, its definitions may be identical or different,
A is N or C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, (C₁-C₄)-alkyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to pentasubstituted by fluorine,
R⁸ is hydrogen, methyl or trifluoromethyl
   and
R¹² is hydrogen,
   and the salts, solvates and solvates of the salts thereof.

Preference is also given to compounds of the formula (I) in which
R¹ is halogen, cyano or (C₁-C₄)-alkyl,
R² is a substituent selected from the group of halogen, cyano, (C₁-C₆)-alkyl and (C₁-C₆)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to pentasubstituted by fluorine,
n is 0, 1 or 2,
   where, in the case that the substituent R² occurs twice, its definitions may be the same or different,
A is C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to pentasubstituted by fluorine,
R⁸ is hydrogen, methyl or trifluoromethyl
   and
R¹² is fluorine,
   and the salts, solvates and solvates of the salts thereof.

In the context of the present invention, particular preference is given to compounds of the formula (I) in which
R¹ is fluorine, chlorine, bromine, cyano or (C₁-C₄)-alkyl,
R² is a substituent selected from the group of fluorine, chlorine, bromine, cyano, (C₁-C₄)-alkyl and (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
n is 0, 1 or 2,
   where, in the case that the substituent R² occurs twice its definitions may be the same or different,
A is N or C—R⁷,
R³ is hydrogen or fluorine,
R⁴ is hydrogen, fluorine, chlorine or methyl,
R⁵ is hydrogen, fluorine, chlorine, cyano, trifluoromethyl, (C₁-C₄)-alkyl, trifluoromethoxy or (C₁-C₄)-alkoxy,
R⁶ and R⁷ are the same or different and are each independently hydrogen, fluorine, chlorine, bromine, cyano, (C₁-C₄)-alkyl or (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
R⁸ is hydrogen, methyl or trifluoromethyl
   and
R¹² is hydrogen,
   and the salts, solvates and solvates of the salts thereof.

Particular preference is also given to compounds of the formula (I) in which
R¹ is fluorine, chlorine, bromine, cyano or (C₁-C₄)-alkyl,
R² is a substituent selected from the group of fluorine, chlorine, bromine, cyano, (C₁-C₄)-alkyl, trifluoromethyl, (C₁-C₄)-alkoxy and trifluoromethoxy,
n is 0, 1 or 2,
   where, in the case that the substituent R² occurs twice, its definitions may be the same or different,
A is C—R⁷,
R³ is hydrogen or fluorine,
R⁴ is hydrogen, fluorine, chlorine or methyl,
R⁵ is hydrogen, fluorine, chlorine, cyano, trifluoromethyl, (C₁-C₄)-alkyl, trifluoromethoxy or (C₁-C₄)-alkoxy,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
R⁸ is hydrogen, methyl or trifluoromethyl
   and
R¹² is fluorine,
   and the salts, solvates and solvates of the salts thereof.

Of particular significance in the context of the present invention are compounds of the formula (I) in which

R¹ is fluorine, chlorine, bromine, cyano or methyl,
and the salts, solvates and solvates of the salts thereof.

Equally of particular significance in the context of the present invention are compounds of the formula (I) in which

R³ and R⁴ are each independently hydrogen or fluorine,
and the salts, solvates and solvates of the salts thereof.

Equally of particular significance in the context of the present invention are compounds of the formula (I) in which

R⁵ is hydrogen, fluorine, chlorine, methyl or trifluoromethyl,
and the salts, solvates and solvates of the salts thereof.

In the context of the present invention, very particular preference is given to compounds of the formula (I) in which
R¹ is fluorine, chlorine, bromine, cyano or methyl,
R² is a substituent selected from the group of fluorine, chlorine, bromine, cyano, (C₁-C₄)-alkyl, trifluoromethyl, (C₁-C₄)-alkoxy and trifluoromethoxy,
n is 0, 1 or 2,
   where, in the case that the substituent R² occurs twice, its definitions may be the same or different,
A is C—R⁷,
R³ is hydrogen,
R⁴ is hydrogen or fluorine,
R⁵ is hydrogen, fluorine, chlorine, methyl or trifluoromethyl,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
R⁸ is hydrogen or trifluoromethyl
   and
R¹² is hydrogen,
   and the salts, solvates and solvates of the salts thereof.

Very particular preference is also given to compounds of the formula (I) in which
R¹ is fluorine, chlorine or cyano,
R² is a substituent selected from the group of fluorine, chlorine, (C₁-C₄)-alkoxy and trifluoromethoxy,
n is 0 or 1,
A is C—R⁷,
R³ and R⁴ are each hydrogen,
R⁵ is hydrogen, fluorine, chlorine, methyl or trifluoromethyl,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
R⁸ is hydrogen
   and
R¹² is fluorine,
   and the salts, solvates and solvates of the salts thereof.

The radical definitions specified individually in the particular combinations or preferred combinations of radicals are, irrespective of the particular combinations of the radicals specified, also replaced as desired by radical definitions of other combinations.

Very particular preference is given to combinations of two or more of the abovementioned preferred ranges.

The invention further provides a process for preparing the inventive compounds of the formula (I), characterized in that a compound of the formula (II)

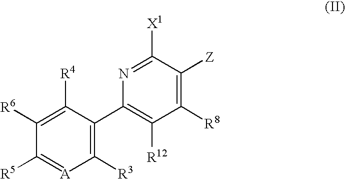

in which A, R³, R⁴, R⁵, R⁶, R⁸ and R¹² are each as defined above,
X¹ is a suitable leaving group, for example halogen, especially chlorine,
and
Z is the —CHO, —CONH₂, —CN or —COOR¹¹ group in which
R¹¹ is (C₁-C₄)-alkyl,
   in an inert solvent in the presence of a base, is reacted with a compound of the formula (III)

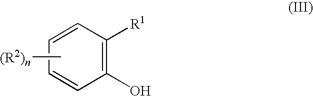

in which R¹, R² and n are each as defined above
to give compounds of the formula (IV)

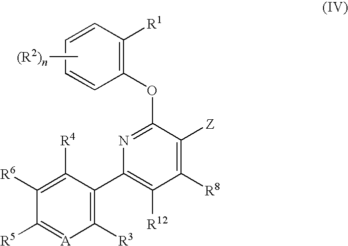

in which A, R¹, R², R³, R⁴, R⁵, R⁶, R⁸, R¹², Z and n are defined as specified above,
and these compounds are converted to the carboxylic acids of the formula (I) by oxidation when Z is —CHO, or by basic or acidic hydrolysis when Z is —CN or —COOR¹¹, or by acidic or basic hydrolysis or by reaction with sodium nitrite in an acetic acid/acetic anhydride mixture and subsequent treatment with hydrochloric acid when Z is —CONH₂, and the compounds of the formula (I) are optionally reacted with the corresponding (i) solvents and/or (ii) bases or acids to give their solvates, salts and/or solvates of the salts.

The compounds of the formula (II) can be prepared by coupling compounds of the formula (V)

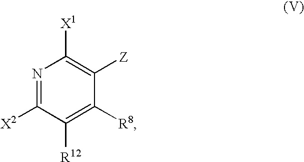

in which R⁸, R¹² and Z are each as defined above and
X¹ and X² are the same or different and are each a suitable leaving group, for example halogen, especially chlorine,
   in an inert solvent in the presence of a suitable transition metal catalyst and optionally of a base, with a compound of the formula (VI)

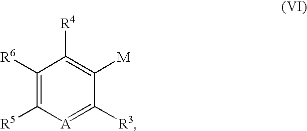

in which A, R³, R⁴, R⁵ and R⁶ are each as defined above and
M is the —B(OH)₂, —ZnHal or —MgHal group in which
Hal is halogen, especially chlorine, bromine or iodine.

Some compounds of the formula (II), in which Z is cyano are also commercially available or known from the literature [see, for example, Zhurnal Organicheskoi Khimii 22 (5), 1061-1065 (1986); J. Med. Chem. 14 (4), 339-344 (1971)].

The compounds of the formulae (III), (V) and (VI) are commercially available, known from the literature or can be prepared in analogy to literature processes. In the case of an organozinc compound of the formula (VI) [M=ZnHal], it can optionally also be obtained in situ from the corresponding Grignard compound [M=MgHal] and a zinc halide [cf., for example, Fu et al., J. Am. Chem. Soc. 123, 2719-2724 (2001)].

Inert solvents of the process step (II)+(III)→(IV) are, for example, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as dimethylformamide, dimethyl sulfoxide, N,N′-dimethylpropyleneurea (DMPU), N-methyl-pyrrolidinone (NMP), pyridine, acetone, 2-butanone or acetonitrile. It is equally possible to use mixtures of the solvents mentioned. Preference is given to using dimethylformamide or toluene.

Suitable bases for the process step (II)+(III)→(IV) are customary inorganic bases. These include especially alkali metal hydroxides, for example lithium hydroxide, sodium hydroxide or potassium hydroxide, alkali metal or alkaline earth metal carbonates such as lithium carbonate, sodium carbonate, potassium carbonate, calcium carbonate or cesium carbonate, or alkali metal hydrides such as sodium hydride or potassium hydride. Preference is given to potassium carbonate or cesium carbonate. The base is used here in an amount of from 1 to 5 mol, preferably in an amount of from 1.2 to 3 mol, based on 1 mol of the compound of the formula (III).

The phenyl ether synthesis (II)+(III)→(IV) can optionally also advantageously be performed with the aid of a palladium catalyst, for example with palladium(II) acetate in combination with a phosphine ligand such as 2-(di-tert-butylphosphino)-1,1′-binaphthyl.

The reaction (II)+(III)→(IV) is effected generally within a temperature range from 0° C. to +150° C., preferably at from +20° C. to +120° C. The reaction can be performed at standard, elevated or reduced pressure (for example from 0.5 to 5 bar). In general, standard pressure is employed.

The hydrolysis of the carboxylic ester in process step (IV) [Z═COOR¹¹]→(I) is effected by customary methods by treating the esters with acids or bases in inert solvents, and the salts formed initially in the latter case are converted to the free carboxylic acids by subsequent treatment with acids. In the case of the tert-butyl esters, the ester cleavage is effected preferably with acids.

Suitable inert solvents for the hydrolysis of the carboxylic esters are water or the organic solvents customary for an ester cleavage. These include especially alcohols such as methanol, ethanol, n-propanol, isopropanol, n-butanol or tert-butanol, ethers such as diethyl ether, tetrahydrofuran, dioxane or glycol dimethyl ether, or other solvents such as acetone, acetonitrile, dichloromethane, dimethylformamide or dimethyl sulfoxide. It is equally possible to use mixtures of the solvents mentioned. In the case of a basic ester hydrolysis, preference is given to using mixtures of water with dioxane, tetrahydrofuran, methanol and/or ethanol. In the case of the reaction with trifluoroacetic acid, preference is given to using dichloromethane, and, in the case of the reaction with hydrogen chloride, preference is given to using tetrahydrofuran, diethyl ether, dioxane or water.

Suitable bases for the ester hydrolysis are the customary inorganic bases. These include especially alkali metal or alkaline earth metal hydroxides, for example sodium hydroxide, lithium hydroxide, potassium hydroxide or barium hydroxide, or alkali metal or alkaline earth metal carbonates such as sodium carbonate, potassium carbonate or calcium carbonate. Preference is given to using sodium hydroxide or lithium hydroxide.

Suitable acids for the ester cleavage are generally sulfuric acid, hydrogen chloride/hydrochloric acid, hydrogen bromide/hydrobromic acid, phosphoric acid, acetic acid, trifluoroacetic acid, toluenesulfonic acid, methanesulfonic acid or trifluoromethanesulfonic acid or mixtures thereof, optionally with addition of water. Preference is given to hydrogen chloride or trifluoroacetic acid in the case of the tert-butyl esters, and hydrochloric acid in the case of the methyl esters.

The esters are cleaved generally within a temperature range from 0° C. to +100° C., preferably at from 0° C. to +50° C. The reaction can be performed at standard, elevated or reduced pressure (for example from 0.5 to 5 bar). In general, standard pressure is employed.

The hydrolysis of the carbonitriles in process step (IV) [Z═CN]→(I) is effected in an analogous manner by reacting the nitriles under hot conditions with strong bases, preferably aqueous or ethanolic potassium hydroxide solution, or strong acids, preferably aqueous sulfuric acid.

The conversion of the primary carboxamides of the formula (IV) [Z═CONH₂] to the carboxylic acids of the formula (I) is equally effected by customary processes by acidic or basic hydrolysis or preferably by reaction with sodium nitrite in an acetic acid/acetic anhydride mixture and subsequent treatment with hydrochloric acid.

The oxidation of the aldehydes of the formula (IV) [Z═CHO] to the carboxylic acids of the formula (I) is effected by methods customary in the literature, for example by reacting with potassium permanganate or chromium(VI) reagents, with hydrogen peroxide, for example in the presence of urea, or preferably with sodium chlorite in the presence of, for example, potassium dihydrogen phosphate or amidosulfonic acid.

Transition metal catalysts, catalyst ligands and auxiliary bases for the coupling reactions (V)+(VI)→(II) are known from the literature [cf., for example, J. Hassan et al., Chem. Rev. 102, 1359-1469 (2002)] and commercially available. Preference is given to using palladium catalysts or nickel catalysts.

In the case of boronic acid coupling [M=B(OH)₂ in (VI)], the reaction is effected in the presence of an auxiliary base and optionally of an additional catalyst ligand. Preference is given here to using bis(triphenylphosphine)palladium(II) chloride as the catalyst, tris(o-tolyl)phosphine as the further ligand and aqueous potassium carbonate solution as the auxiliary base. In the case of organozinc compounds [M=ZnHal in (VI)], preference is given to using tetrakis(triphenyl-phosphine)palladium(0) as the catalyst.

Inert solvents for the boronic acid coupling (V)+(VI) [M=B(OH)₂]→(II) are, for example, alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, hydrocarbons such as benzene, toluene, xylene, hexane, cyclohexane or mineral oil fractions, or other solvents such as dimethylformamide, dimethyl sulfoxide, N,N′-dimethylpropyleneurea (DMPU), N-methylpyrrolidone (NMP), pyridine, acetonitrile or else water. It is equally possible to use mixtures of the solvents mentioned. Preference is given to using dimethylformamide or dioxane.

The coupling reactions (V)+(VI)→(II) are effected generally within a temperature range from −20° C. to +150° C., preferably at from 0° C. to +80° C. The reactions can be performed at standard, elevated or reduced pressure (for example from 0.5 to 5 bar). In general, standard pressure is employed.

The preparation of the inventive compounds can be illustrated by the following synthesis schemes:

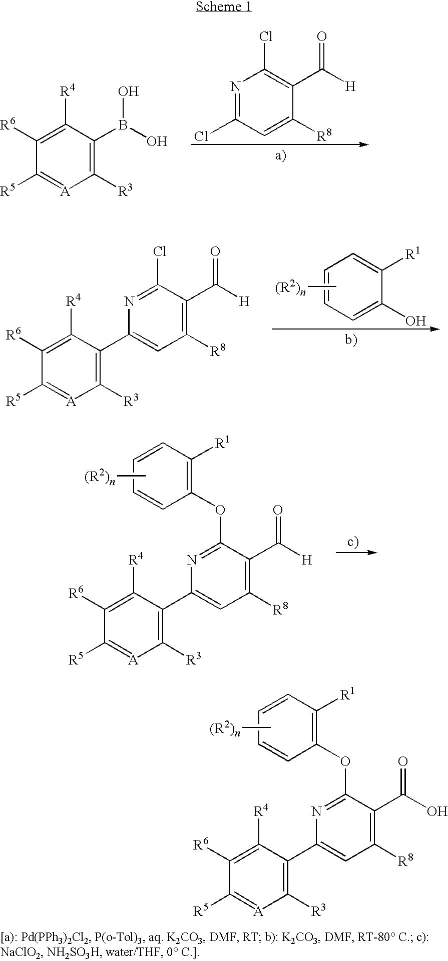

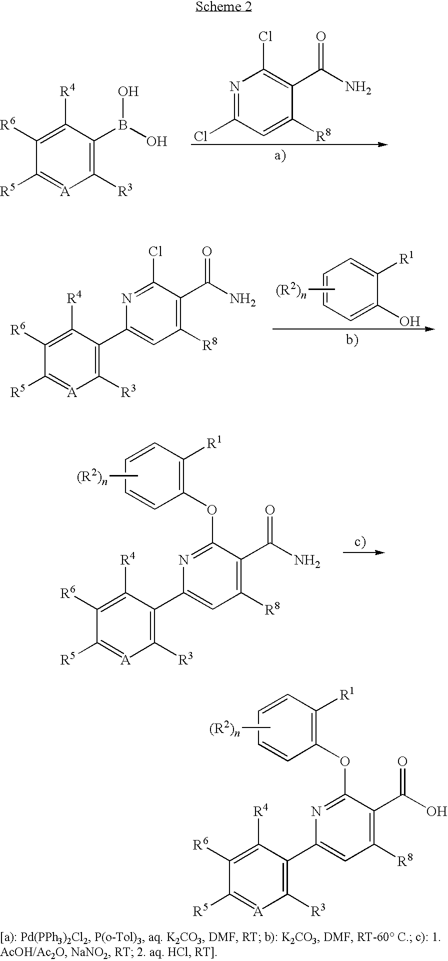

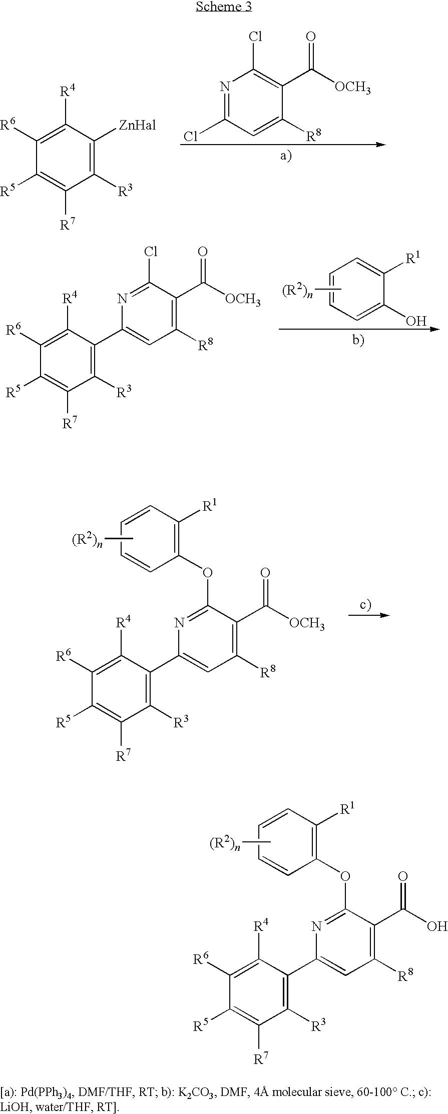

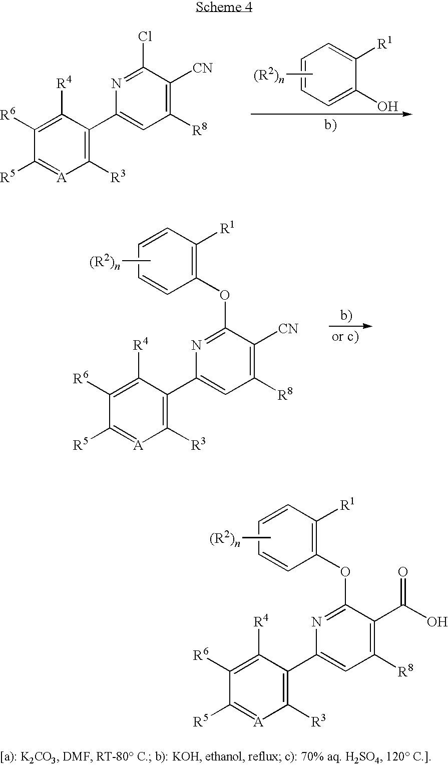

The inventive compounds have valuable pharmacological properties and can be used for the prevention and treatment of disorders in humans and animals.

The inventive compounds are highly active PPAR-alpha modulators and are suitable as such especially for the primary and/or secondary prevention and treatment of cardiovascular disorders which are caused by disruptions in the fatty acid and glucose metabolism. Such disorders include dyslipidemias (hypercholesterolemia, hypertriglyceridemia, elevated concentrations of the postprandial plasma triglycerides, hypoalphalipoproteinemia, combined hyperlipidemias), arteriosclerosis and metabolic disorders (metabolic syndrome, hyperglycemia, insulin-dependent diabetes, non-insulin-dependent diabetes, gestation diabetes, hyperinsulinemia, insulin resistance, glucose intolerance, adiposity and diabetic late complications such as retinopathy, nephropathy and neuropathy).

As highly active PPAR-alpha modulators, the inventive compounds are suitable especially also for the primary and/or secondary prevention and treatment of heart failure.

In the context of the present invention, the term “heart failure” also encompasses more specific or related disease forms such as right heart failure, left heart failure, global failure, ischemic cardiomyopathy, dilatative cardiomyopathy, congenital heart defects, heart valve defects, heart failure in the event of heart valve defects, mitral valve stenosis, mitral valve failure, aortic valve stenosis, aortic valve failure, tricuspidal stenosis, tricuspidal failure, pulmonary valve stenosis, pulmonary valve failure, combined heart valve defects, heart muscle inflammation (myocarditis), chronic myocarditis, acute myocarditis, viral myocarditis, diabetic heart failure, alcohol-toxic cardiomyopathy, cardiac storage disorders, diastolic heart failure and systolic heart failure.

Further independent risk factors for cardiovascular disorders which can be treated by the inventive compounds are hypertension, ischemia, myocardial infarction, angina pectoris, heart muscle weakness, restenosis, pulmonary hypertension, increased levels of fibrinogen and of low-density LDL and elevated concentrations of plasminogen activator inhibitor 1 (PAI-1).

Furthermore, the inventive compounds may also be used for the treatment and/or prevention of micro- and macrovascular damage (vasculitis), reperfusion damage, arterial and venous thromboses, edemas, cancers (skin cancer, liposarcomas, carcinomas of the gastrointestinal tract, of the liver, pancreas, lung, kidney, ureter, prostate and of the genital tract), of disorders of the central nervous system and neurodegenerative disorders (stroke, Alzheimer's disease, Parkinson's disease, dementia, epilepsy, depression, multiple sclerosis), of inflammatory disorders, immune disorders (Crohn's disease, ulcerative colitis, lupus erythematosus, rheumatoid arthritis, asthma), kidney disorders (glomerulonephritis), thyroid disorders (hyperthyreosis), disorders of the pancreas (pancreatitis), liver fibrosis, skin disorders, (psoriasis, acne, eczema, neurodermitis, dermatitis, keratitis, scar formation, wart formation, chillblains), viral disorders (HPV, HCMV, HIV), cachexia, osteoporosis, gout, incontinence, and for wound healing and angiogenesis.

The efficacy of the inventive compounds can be tested, for example, in vitro by the transactivation assay described in the example part.

The efficacy of the inventive compounds in vivo can be tested, for example, by the studies described in the example part.

The present invention further provides for the use of the inventive compounds for the treatment and/or prevention of disorders, especially of the aforementioned disorders.

The present invention further provides for the use of the inventive compounds for producing a medicament for the treatment and/or prevention of disorders, especially of the aforementioned disorders.

The present invention further provides a process for the treatment and/or prevention of disorders, especially of the aforementioned disorders, using an effective amount of at least one of the inventive compounds.

The inventive compounds may be used alone or, if required, in combination with other active ingredients. The present invention further provides medicaments comprising at least one of the inventive compounds and one or more further active ingredients, especially for the treatment and/or prevention of the aforementioned disorders.

Suitable active ingredients for combinations include, by way of example and with preference: substances which modify lipid metabolism, antidiabetics, hypotensives, perfusion-enhancing and/or antithrombotic agents, and also antioxidants, chemokine receptor antagonists, p38-kinase inhibitors, NPY agonists, orexin agonists, anorectics, PAF-AH inhibitors, antiphlogistics (COX inhibitors, LTB₄-receptor antagonists), analgesics (aspirin), antidepressants and other psychopharmaceuticals.

The present invention provides especially combinations comprising at least one of the inventive compounds and at least one lipid metabolism-modifying active ingredient, an antidiabetic, an active hypotensive ingredient and/or an antithrombotic agent.

The inventive compounds can preferably be combined with one or more
lipid metabolism-modifying active ingredients, by way of example and with preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbers, bile acid reabsorption inhibitors, MTP inhibitors, lipase inhibitors, LpL activators, fibrates, niacin, CETP inhibitors, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavengers,
antidiabetics mentioned in the Rote Liste 2004/II, chapter 12, and also, by way of example and with preference, those from the group of the sulfonylureas, biguanides, meglitinide derivatives, glucosidase inhibitors, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861,
active hypotensive ingredients, by way of example and with preference from the group of the calcium antagonists, angiotensin AII antagonists, ACE inhibitors, beta-receptor blockers, alpha-receptor blockers, ECE inhibitors and the vasopeptidase inhibitors;
antithrombotic agents, by way of example and with preference from the group of the platelet aggregation inhibitors or the anticoagulants;
diuretics;
aldosterone and mineral corticoid receptor antagonists;
vasopressin receptor antagonists;
organic nitrates and NO donors;
positive-inotropically active ingredients;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, in particular PDE 5 inhibitors such as sildenafil, vardenafil and tadalafil, and PDE 3 inhibitors such as milrinone;
natriuretic peptides such as for example “atrial natriuretic peptide” (ANP, anaritide), “B-type natriuretic peptide” or “brain natriuretic peptide” (BNP, nesiritide), “C-type natriuretic peptide” (CNP) and urodilatin;
calcium sensitizers, by way of example and with preference levosimendan;
potassium supplements;
NO-independent but heme-dependent stimulators of guanylate cyclase, especially the compounds described in WO 00/06568, WO 00/06569, WO 02/42301 and WO 03/095451;
NO- and heme-independent activators of guanylate cyclase, especially the compounds described in WO 01/19355, WO 01/19776, WO 01/19778, WO 01/19780, WO 02/070462 and WO 02/070510;
inhibitors of human neutrophil elastase (FINE), for example sivelestat or DX-890 (reltran);
compounds inhibiting the signal transduction cascade, for example tyrosine kinase inhibitors, in particular sorafenib, imatinib, gefitinib and erlotinib; and/or
compounds influencing the energy metabolism of the heart, for example etomoxir, dichloroacetate, ranolazine or trimetazidine.

Lipid metabolism-modifying active ingredients are preferably understood to mean compounds from the group of the HMG-CoA reductase inhibitors, squalene synthesis inhibitors, ACAT inhibitors, cholesterol absorption inhibitor, MTP inhibitors, lipase inhibitors, thyroid hormones and/or thyroid mimetics, niacin receptor agonists, CETP inhibitors, PPAR-gamma agonists, PPAR-delta agonists, polymeric bile acid adsorbers, bile acid reabsorption inhibitors, antioxidants/radical scavengers and also the cannabinoid receptor 1 antagonists.

In a preferred embodiment of the invention, the inventive compounds are administered in combination with an HMG-CoA reductase inhibitor from the class of the statins, by way of example and with preference lovastatin, simvastatin, pravastatin, fluvastatin, atorvastatin, rosuvastatin, cerivastatin or pitavastatin.

In a preferred embodiment of the invention, the inventive compounds are administered in combination with a squalene synthesis inhibitor, by way of example and with preference BMS-188494 or TAK-475.

In a preferred embodiment of the invention, the inventive compounds are administered in combination with an ACAT inhibitor, by way of example and with preference melinamide, pactimibe, eflucimibe or SMP-797.

In a preferred embodiment of the invention, the compounds according to the invenetion are administered in combination with a cholesterol absorption inhibitor, by way of example and with preference ezetimibe, tiqueside or pamaqueside.

In a preferred embodiment of the invention, the inventive compounds are administered in combination with an MTP inhibitor, by way of example and with preference implitapide or JTT-130.

In a preferred embodiment of the invention, the inventive compounds are administered in combination with a lipase inhibitor, by way of example and with preference orlistat.

In a preferred embodiment of the invention, the inventive compounds are administered in combination with a thyroid hormone and/or thyroid mimetic, by way of example and with preference D-thyroxine or 3,5,3′-triiodothyronine (T3).

In a preferred embodiment of the invention, the inventive compounds are administered in combination with an agonist of the niacin receptor, by way of example and with preference niacin, acipimox, acifran or radecol.

In a preferred embodiment of the invention, the inventive compounds are administered in combination with a CETP inhibitor, by way of example and with preference torcetrapib, JTT-705 or CETP vaccine (Avant).

In a preferred embodiment of the invention, the inventive compounds are administered in combination with a PPAR-gamma agonist, by way of example and with preference pioglitazone or rosiglitazone.

In a preferred embodiment of the invention, the inventive compounds are administered in combination with a PPAR-delta agonist, by way of example and with preference GW-501516.

In a preferred embodiment of the invention, the inventive compounds are administered in combination with a polymeric bile acid adsorber, by way of example and with preference cholestyramine, colestipol, colesolvam, CholestaGel or colestimide.

In a preferred embodiment of the invention, the inventive compounds are administered in combination with a bile acid reabsorption inhibitor, by way of example and with preference ASBT (=IBAT) inhibitors, such as, for example, AZD-7806, S-8921, AK-105, BARI-1741, SC-435 or SC-635.

In a preferred embodiment of the invention, the inventive compounds are administered in combination with a antioxidant/radical scavenger, by way of example and with preference probucol, AGI-1067, BO-653 or AEOL-10150.

In a preferred embodiment of the invention, the inventive compounds are administered in combination with a cannabinoid receptor 1 antagonist, by way of example and with preference rimonabant or SR-147778.

Antidiabetics are preferably understood to mean insulin and insulin derivatives, and also orally active hypoglycemic acid compounds. Here, insulin and insulin derivatives include both insulins of animal, human or biotechnological origin and also mixtures thereof. The orally active hypoglycemic active ingredients preferably include sulfonylureas, biguanides, meglitinide derivatives, glucosidase inhibitors and PPAR-gamma agonists.

In a preferred embodiment of the invention, the inventive compounds are administered in combination with insulin.

In a preferred embodiment of the invention, the inventive compounds are administered in combination with a sulfonylurea, by way of example and with preference tolbutamide, glibenclamide, glimepiride, glipizide or gliclazide.

In a preferred embodiment of the invention, the inventive compounds are administered in combination with a biguanide, by way of example and with preference metformin

In a preferred embodiment of the invention, the inventive compounds are administered in combination with a meglitinide derivative, by way of example and with preference repaglinide or nateglinide.

In a preferred embodiment of the invention, the inventive compounds are administered in combination with a glucosidase inhibitor, by way of example and with preference miglitol or acarbose.

In a preferred embodiment of the invention, the inventive compounds are administered in combination with a PPAR-gamma agonist, for example from the class of the thiazolidinediones, by way of example and with preference pioglitazone or rosiglitazone.

The hypotensive agents are preferably understood to mean compounds from the group of the calcium antagonists, angiotensin AII antagonists, ACE inhibitors, beta-receptor blockers, alpha-receptor blockers and of the diuretics.

In a preferred embodiment of the invention, the inventive compounds are administered in combination with a diuretic, by way of example and with preference a loop diuretic such as furosemide, bumetanide or torsemide, or a thiazide or thiazide-like diuretic such as chlorothiazide or hydrochlorothiazide.

In a preferred embodiment of the invention, the inventive compounds are administered in combination with an aldosterone or mineral corticoid receptor antagonist, by way of example and with preference spironolactone or eplerenone.

In a preferred embodiment of the invention, the inventive compounds are administered in combination with a vasopressin receptor antagonist, by way of example and with preference conivaptan, tolvaptan, lixivaptan or SR-121463.

In a preferred embodiment of the invention, the inventive compounds are administered in combination with an organic nitrate or NO donor, by way of example and with preference sodium nitroprusside, nitroglycerine, isosorbide mononitrate, isosorbide dinitrate, molsidomine or SIN-1, or in combination with inhalative NO.

In a preferred embodiment of the invention, the inventive compounds are administered in combination with a positively-inotropically active compound, by way of example and with preference cardiac glycosides (digoxin), beta-adrenergic and dopaminergic agonists such as isoproterenol, adrenalin, noradrenalin, dopamine or dobutamine.

In a preferred embodiment of the invention, the inventive compounds are administered in combination with a calcium antagonist, by way of example and with preference nifedipine, amlodipine, verapamil or diltiazem.

In a preferred embodiment of the invention, the inventive compounds are administered in combination with an angiotensin AII antagonist, by way of example and with preference losartan, valsartan, candesartan, embusartan or telmisartan.

In a preferred embodiment of the invention, the inventive compounds are administered in combination with an ACE inhibitor, by way of example and with preference enalapril, captopril, ramipril, delapril, fosinopril, quinopril, perindopril or trandopril.

In a preferred embodiment of the invention, the inventive compounds are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an alpha-receptor blocker, by way of example and with preference prazosin.

In a preferred embodiment of the invention, the inventive compounds are administered in combination with antisympathotonics, by way of example and with preference reserpine, clonidine or alpha-methyldopa, or in combination with potassium channel agonists, by way of example and with preference minoxidil, diazoxide, dihydralazine or hydralazine.

Antithrombotics are preferably understood to mean compounds from the group of the platelet aggregation inhibitors or of the anticoagulants.

In a preferred embodiment of the invention, the inventive compounds are administered in combination with a platelet aggregation inhibitor, by way of example and with preference aspirin, clopidogrel, ticlopidine or dipyridamol.

In a preferred embodiment of the invention, the inventive compounds are administered in combination with a thrombin inhibitor, by way of example and with preference ximelagatran, melagatran, bivalirudin or clexane.

In a preferred embodiment of the invention, the inventive compounds are administered in combination with a GPIIb/IIIa antagonist, by way of example and with preference tirofiban or abciximab.

In a preferred embodiment of the invention, the inventive compounds are administered in combination with a factor Xa inhibitor, by way of example and with preference rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the inventive compounds are administered in combination with heparin or a low molecular weight (LMW) heparin derivative.

In a preferred embodiment of the invention, the inventive compounds are administered in combination with a vitamin K antagonist, by way of example and with preference coumarin.

In the context of the present invention, particular preference is given to combinations comprising at least one of the inventive compounds and one or more further active ingredients selected from the group consisting of HMG-CoA reductase inhibitors (statins), diuretics, beta-receptor blockers, organic nitrates and NO donors, ACE inhibitors, angiotensin AII antagonists, aldosterone receptor and mineralocorticoid receptor antagonists, vasopressin receptor antagonists, platelet aggregation inhibitors and anticoagulants, and to the use thereof for the treatment and/or prevention of the aforementioned disorders.

The present invention further provides medicaments which comprise at least one inventive compound, typically together with one or more inert, non-toxic, pharmaceutically suitable excipients, and the use therefore for the aforementioned purposes.

The inventive compounds can act systemically and/or locally. For this purpose, they can be administered in a suitable manner, for example orally, parenterally, pulmonally, nasally, sublingually, lingually, buccally, rectally, dermally, transdermally, conjunctivally, otically, or as an implant or stent.

For these administration routes, the inventive compounds can be administered in suitable administration forms.

Suitable for oral administration are administration forms which work in accordance with the prior art and release the inventive compounds rapidly and/or in modified form and which comprise the inventive compounds in crystalline and/or amorphicized and/or dissolved form, for example tablets (uncoated or coated tablets, for example with enteric coats or coats which dissolve in a delayed manner or are insoluble and which control the release of the inventive compounds), films/wafers or tablets which dissolve rapidly in the oral cavity, films/lyophilizates, capsules (for example hard or soft gelatin capsules), sugar-coated tablets, granules, pellets, powders, emulsions, suspensions, aerosols or solutions.

Parenteral administration may take place with avoidance of a bioabsorption step (for example intravenously, intraarterially, intracardially, intraspinally or intralumbarly), or with bioabsorption (for example intramuscularly, subcutaneously, intracutaneously, percutaneously or intraperitoneally). Administration forms suitable for parenteral administration are inter alia preparations for injection or infusion in the form of solutions, suspensions, emulsions, lyophilizates or sterile powders.

Suitable for other administration routes are, for example, medicaments suitable for inhalation (inter alia powder inhalers, nebulizers), nose drops, solutions or sprays, tablets to be administered lingually, sublingually or buccally, films/wafers or capsules, suppositories, preparations to be administered to ears or eyes, vaginal capsules, aqueous suspensions (lotions, shaking mixtures), lipophilic suspensions, ointments, creams, transdermal therapeutic systems (for example plasters), milk, pastes, foams, powders for pouring, implants or stents.

Preference is given to oral or parenteral administration, in particular to oral and intravenous administration.

The inventive compounds can be converted into the administration forms mentioned. This can be carried out in a manner known per se by mixing with inert non-toxic pharmaceutically suitable auxiliaries. These auxiliaries include inter alia carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, for example ascorbic acid), colorants (for example inorganic pigments, for example iron oxides), and flavor and/or odor corrigents.

In general, it has been found to be advantageous in the case of parenteral administration to administer amounts of about 0.001 to 1 mg/kg, preferably about 0.01 to 0.5 mg/kg of body weight to obtain effective results. In the case of oral administration, the dosage is from about 0.01 to 100 mg/kg, preferably from about 0.01 to 20 mg/kg and very particularly preferably from 0.1 to 10 mg/kg of body weight.

In spite of this, it may be necessary to deviate from the amounts mentioned, namely depending on body weight, administration route, individual response to the active compound, the type of preparation and the time or the interval at which administration takes place. Thus, in some cases it may be sufficient to administer less than the abovementioned minimum amount, whereas in other cases the upper limit mentioned has to be exceeded. In the case of the administration of relatively large amounts, it may be expedient to divide these into a plurality of individual doses which are administered over the course of the day.

The working examples below illustrate the invention. The invention is not limited to the examples.

The percentages in the tests and examples below are, unless stated otherwise, percentages by weight; parts are parts by weight. Solvent ratios, dilution ratios and concentrations of liquid/liquid solutions are in each case based on volume.

### A. EXAMPLES

### Abbreviations

Ac₂O acetic anhydride
AcOH acetic acid
aq. aqueous
br. broad (in NMR)
TLC thin-layer chromatography
DCI direct chemical ionization (in MS)
DCM dichloromethane
DMF dimethylformamide
DMSO dimethyl sulfoxide
EI electron impact ionization (in MS)
eq. equivalent(s)
ESI electrospray ionization (in MS)
h hour(s)
Hal halogen
HPLC high-pressure, high-performance liquid chromatography
LC-MS liquid chromatography-coupled mass spectrometry
min minute(s)
MS mass spectrometry
m_{z} centered multiplet (in NMR)
NMR nuclear magnetic resonance spectrometry
o-Tol ortho-tolyl
Ph phenyl
RP reverse phase (in HPLC)
RT room temperature
Rₜ retention time (in HPLC)
THF tetrahydrofuran
UV ultraviolet spectrometry
v/v volume-to-volume ratio (of a mixture)

### LC-MS and HPLC Methods:

### Method 1 (LC-MS):

Instrument type MS: Micromass ZQ; Instrument type HPLC: HP 1100 series; UV DAD; column: Phenomenex Gemini 3μ, 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm

### Method 2 (LC-MS):

Instrument type MS: Micromass ZQ; Instrument type HPLC: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm

### Method 3 (LC-MS):

Instrument: Micromass Quattro LCZ with HPLC Agilent series 1100; column: Phenomenex Onyx Monolithic C18, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow rate: 2 ml/min; oven: 40° C.; UV detection: 208-400 nm

### Method 4 (LC-MS):

Instrument: Micromass Quattro LCZ with HPLC Agilent series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm

### Method 5 (LC-MS):

Instrument type MS: Waters ZQ; Instrument type HPLC: Waters Alliance 2795; column: Merck Chromolith RP18e, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow rate: 2 ml/min; oven: 40° C.; UV detection: 210 nm

### Method 6 (LC-MS):

Instrument: Micromass Quattro LCZ with HPLC Agilent series 1100; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm

### Method 7 (LC-MS):

Instrument type MS: Micromass ZQ; Instrument type HPLC: HP 1100 series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm

Method 8 (preparative HPLC):

Instrument: Abimed Gilson Pump 305/306, Manometric Module 806; column: Grom-Sil C18 10 μm, 250 mm×30 mm; eluent: A=water, B=acetonitrile; gradient 0.0 min 10% B→3 min 10% B→30 min 95% B→42 min 95% B→42.1 min 10% B→45 min 10% B; flow rate: 50 ml/min; column temperature: RT; UV detection: 210 nm

Method 9 (preparative HPLC):

Instrument: Abimed Gilson Pump 305/306, Manometric Module 806; column: Grom-Sil 1200DS-4HE 10 μm, 250 mm×40 mm; eluent: A=water, B=acetonitrile; gradient 0.0 min 10% B→3 min 10% B→27 min 98% B→34 min 98% B→34.01 min 10% B→38 min 10% B; flow rate: 50 ml/min; column temperature: RT; UV detection: 214 nm

### Method 10 (Preparative HPLC):

Instrument: Abimed Gilson Pump 305/306, Manometric Module 806; column: Grom-Sil 1200DS-4HE 10 μm, 250 mm×40 mm; eluent: A=water+0.75 ml formic acid/L water, B=acetonitrile; gradient: 0.0 min 10% B→3 min 10% B→27 min 98% B→34 min 98% B→34.01 min 10% B→38 min 10% B; flow rate: 50 ml/min; column temperature: RT; UV detection: 214 nm

### Method 11 (LC-MS):

Instrument: Micromass Quattro LCZ with HPLC Agilent series 1100; column: Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

### Method 12 (LC-MS):

Instrument type MS: Micromass ZQ; Instrument type HPLC: Waters Alliance 2795; column: Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient 0.0 min 90% A→0.1 min 90% A→3.0 min 5% A→4.0 min 5% A→4.01 min 90% A; flow rate: 2 ml/min; oven: 50° C.; UV detection: 210 nm

Method 13 (preparative HPLC):

Instrument: Abimed Gilson Pump 305/306, Manometric Module 806; column: Grom-Sil 1200DS-4HE 10 μm, 250 mm×40 mm; eluent: A=water, B=acetonitrile; gradient 0.0 min 30% B→5 min 30% B→30 min 95% B→50 min 95% B→51 min 30% B→55 min 30% B; flow rate: 50 ml/min; column temperature: RT; UV detection: 214 nm

### Method 14 (LC-MS):

Instrument: Micromass QuattroPremier with Waters HPLC Acquity; column: Thermo Hypersil GOLD 1.9μ 50 mm×1 mm; eluent A: 1 l water+0.5 ml 50% formic acid, eluent B: 1 l acetonitrile+0.5 ml 50% formic acid; gradient: 0.0 min 90% A→0.1 min 90% A→1.5 min 10% A→2.2 min 10% A; oven: 50° C.; flow rate: 0.33 ml/min; UV detection: 210 nm

### Starting Compounds and Intermediates

### Example 1A

### 2-Chloro-6-[4-(trifluoromethyl)phenyl]nicotinaldehyde

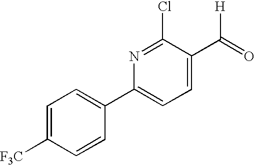

216 mg (1.14 mmol) of 4-(trifluoromethyl)phenylboronic acid and 3.41 ml (6.82 mmol) of a 2 M aqueous potassium carbonate solution are added to 200 mg (1.14 mmol) of 2,6-dichloro-nicotinaldehyde dissolved in 4 ml of DMF. After stirring for 10 min, 159 mg (0.23 mmol) of bis(triphenylphosphine)palladium(II) chloride and 35 mg (0.11 mmol) of tri-2-tolylphosphine are added and the reaction mixture is stirred at RT overnight. After standing at RT for a further two days, for workup, the mixture is first diluted with 10 ml of water and admixed with about 4 ml of 1 N hydrochloric acid, then stirred with 20 ml of ethyl acetate, and filtered through 10 g of Celite. The organic phase is removed and concentrated and the residue is purified by preparative HPLC (method 9). This affords 157 mg (48% of theory) of the target compound.

¹H NMR (400 MHz, DMSO-d₆): δ=7.94 (AA′ part of an AA′BB′ system, 2H), 8.32 (d, 1H), 8.38 (d, 1H), 8.38 (BB′ part of an AA′BB′ system, 2H), 10.32 (s, 1H).

LC-MS (method 2): Rₜ=2.70 min; m/z=286 [M+H]⁺.

### Example 2A

### 2-Chloro-6-[3-(trifluoromethyl)phenyl]nicotinaldehyde

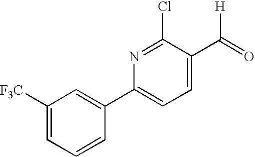

The title compound is prepared and purified analogously to Example 1A. Additional purification is effected by chromatography on silica gel (eluent: 10:1, then 4:1 cyclohexane/ethyl acetate). 200 mg (1.14 mmol) of 2,6-dichloronicotinaldehyde and 216 mg (1.14 mmol) of 3-(trifluoromethyl)phenylboronic acid afford 202 mg (62% of theory) of the target compound.

LC-MS (method 2): Rₜ=2.67 min; m/z=286 [M+H]⁺.

### Example 3A

### 2-Chloro-6-[4-chloro-3-(trifluoromethyl)phenyl]nicotinaldehyde

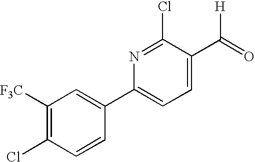

The title compound was prepared and purified analogously to Example 1A, except that double the amount of tri-2-tolylphosphine (69 mg, 0.23 mmol) is used. The total reaction time is about 5 days. 200 mg (1.14 mmol) of 2,6-dichloronicotinaldehyde and 255 mg (1.14 mmol) of 4-chloro-3-(trifluoromethyl)phenylboronic acid afford 139 mg (38% of theory) of the target compound.

¹H NMR (400 MHz, DMSO-d₆): δ=7.94 (d, 1H), 8.38 (AB system, 2H), 8.48 (dd, 1H), 8.55 (d, 1H), 10.31 (s, 1H).

LC-MS (method 3): Rₜ=4.28 min; m/z=338 [M+H+H₂O]⁺, 320 [M+H]⁺.

### Example 4A

### 2-Chloro-6-(4-fluoro-3-methylphenyl)nicotinaldehyde

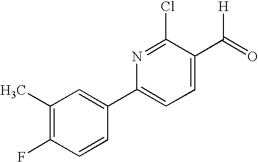

The title compound is prepared and purified analogously to Example 3A. 200 mg (1.14 mmol) of 2,6-dichloronicotinaldehyde and 175 mg (1.14 mmol) of 4-fluoro-3-methylphenylboronic acid afford 100 mg (35% of theory) of the target compound.

¹H NMR (400 MHz, DMSO-d₆): δ=2.34 (s, 3H), 7.33 (t, 1H), 8.05 (ddd, 1H), 8.14 (dd, 1H), 8.19 (d, 1H), 8.30 (d, 1H), 10.29 (s, 1H).

LC-MS (method 2): Rₜ=2.63 min; m/z=250 [M+H]⁺.

### Example 5A

### 2-Chloro-6-(3-fluoro-4-methylphenyl)nicotinaldehyde

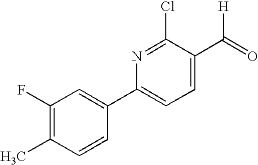

The title compound is prepared and purified analogously to Example 1A. The total reaction time is about 5 days. The product fractions are purified further by another HPLC under the same conditions. 200 mg (1.14 mmol) of 2,6-dichloronicotinaldehyde and 175 mg (1.14 mmol) of 3-fluoro-4-methylphenylboronic acid afford 129 mg (45% of theory) of the target compound.

¹H NMR (500 MHz, DMSO-d₆): δ=2.19 (s, 3H), 7.48 (t, 1H), 7.92 (d, 1H), 7.94 (d, 1H), 8.23 (d, 1H), 8.30 (d, 1H), 10.28 (s, 1H).

LC-MS (method 6): Rₜ=2.72 min; m/z=268 [M+H+H₂O]⁺, 250 [M+H]⁺.

### Example 6A

### 2-Chloro-6-(2,3-difluorophenyl)nicotinaldehyde

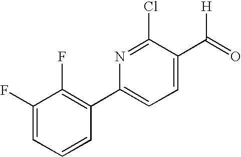

179 mg (1.14 mmol) of 2,3-difluorophenylboronic acid and then 3.4 ml of a 2 M aqueous potassium carbonate solution are added with stirring to a solution of 200 mg (1.14 mmol) of 2,6-dichloropyridine-3-carboxaldehyde in 4 ml of dioxane. After 10 min, 160 mg (0.23 mmol) of bis(triphenylphosphine)palladium(II) chloride and 69 mg (0.23 mmol) of tri-2-tolylphosphine are added and the reaction mixture is then stirred at 60° C. overnight. The mixture is worked up and purified directly by means of preparative HPLC (method 9). This affords 144 mg (50% of theory) of the target compound in a mixture with tri-2-tolylphosphine, which is reacted further in this form.

¹H NMR (400 MHz, DMSO-d₆): δ=7.42 (tdd, 1H), 7.65 (dtd, 1H), 7.80 (ddt, 1H), 8.06 (dd, 1H), 8.39 (d, 1H), 10.31 (s, 1H).

LC-MS (method 1): Rₜ=2.60 min; m/z=254 [M+H]⁺.

### Example 7A

### 2-Chloro-6-(2-chlorophenyl)nicotinaldehyde

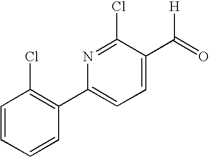

The title compound is prepared and purified anologously to Example 6A starting from 2-chorophenylboronic acid. This affords the target compound in a yield of approx. 28% of theory with an impurity of tri-2-tolylphosphine oxide.

LC-MS (method 3): Rₜ=3.71 min; m/z=252 [M+H]⁺ (tri-2-tolylphosphine oxide: Rₜ=3.67 min; m/z=321 [M+H]⁺).

### Example 8A

### 2-Chloro-6-(2,3-dimethylphenyl)nicotinaldehyde

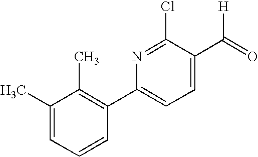

The title compound is prepared and purified analogously to Example 6A starting from 2,3-dimethylphenylboronic acid. This affords the target compound in a yield of 53% of theory.

¹H NMR (400 MHz, DMSO-d₆): δ=2.21 (s, 3H), 2.33 (s, 3H), 7.20-7.28 (m, 2H), 7.31 (dd, 1H), 7.71 (d, 1H), 8.31 (d, 1H), 10.33 (s, 1H).

LC-MS (method 1): Rₜ=2.63 min; m/z=246 [M+H]⁺.

### Example 9A

### 2-Chloro-6-[3-(trifluoromethoxy)phenyl]nicotinaldehyde

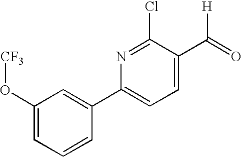

The title compound is prepared and purified analogously to Example 6A proceeding from 3-(trifluoromethoxy)phenylboronic acid. This affords the target compound in a yield of 34% of theory.

¹H NMR (400 MHz, DMSO-d₆): δ=7.59 (br. d, 1H), 7.72 (t, 1H), 8.13 (br. s, 1H), 8.23 (d, 1H), 8.31 (d, 1H), 8.36 (d, 1H), 10.31 (s, 1H).

LC-MS (method 2): Rₜ=2.73 min; m/z=302 [M+H].

### Example 10A

### 2-Chloro-6-(2-fluoro-3-methoxyphenyl)nicotinaldehyde

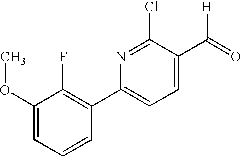

The title compound is prepared and purified analogously to Example 6A starting from 2-fluoro-3-methoxyphenylboronic acid. This affords the title compound in a yield of approx. 31% of theory with an impurity of tri-2-tolylphosphine oxide.

LC-MS (method 1): Rₜ=2.44 min; m/z=284 [M+H+H₂O]⁺, 266 [M+H]⁺ (tri-2-tolylphosphine oxide: Rₜ=2.48 min; m/z=321 [M+H]⁺).

### Example 11A

### 2-(2-Chlorophenoxy)-6-[4-(trifluoromethyl)phenyl]nicotinaldehyde

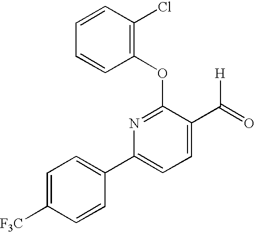

65 mg (0.51 mmol) of 2-chlorophenol and 210 mg (1.52 mmol) of potassium carbonate are added to a solution of 145 mg (0.51 mmol) of 2-chloro-6-[4-(trifluoromethyl)phenyl]nicotinaldehyde from Example 1A in 3 ml of DMF. The mixture is left to stir at RT overnight, then stirred at 80° C. for approx. 4 h to complete the reaction, and, after filtration from the solid, purified by preparative HPLC (method 9). This affords 177 mg (92% of theory) of the target compound.

¹H NMR (400 MHz, DMSO-d₆): δ=7.40 (t, 1H), 7.47-7.58 (m, 2H), 7.68 (t, 1H), 7.82 (d, 2H), 8.03 (d, 2H), 8.04 (d, 1H), 8.41 (d, 1H), 10.50 (s, 1H).

LC-MS (method 2): Rₜ=3.05 min; m/z=378 [M+H]⁺.

### Example 12A

### 2-(2-Chlorophenoxy)-6-[3-(trifluoromethyl)phenyl]nicotinaldehyde

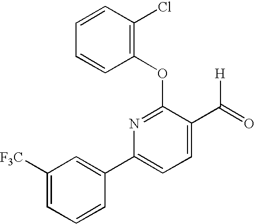

The title compound is prepared and purified analogously to Example 11A. The reaction time at 80° C. is 2 h. Starting from 190 mg (0.61 mmol) of 2-chloro-6-[3-(trifluoromethyl)phenyl]nicotinaldehyde from Example 2A and 78 mg (0.61 mmol) of 2-chlorophenol, 138 mg (90% of theory) of the target compound are obtained.

¹H NMR (400 MHz, DMSO-d₆): δ=7.41 (td, 1H), 7.48-7.58 (m, 2H), 7.65-7.73 (m, 2H), 7.82 (d, 1H), 8.09 (d, 1H), 8.11 (br. s, 1H), 8.20 (d, 1H), 8.40 (d, 1H), 10.50 (s, 1H).

LC-MS (method 2): Rₜ=3.04 min; m/z=378 [M+H]⁺.

### Example 13A

### 2-(2-Chlorophenoxy)-6-[4-chloro-3-(trifluoromethyl)phenyl]nicotinaldehyde

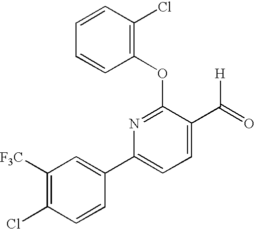

The title compound is prepared and purified analogously to Example 12A. Starting from 135 mg (0.37 mmol) of 2-chloro-6-[4-chloro-3-(trifluoromethyl)phenyl]nicotinaldehyde from Example 3A and 47 mg (0.37 mmol) of 2-chlorophenol, 148 mg (98% of theory) of the target compound are obtained.

¹H NMR (400 MHz, DMSO-d₆): δ=7.41 (td, 1H), 7.47-7.58 (m, 2H), 7.68 (dd, 1H), 7.84 (d, 1H), 8.10 (d, 1H), 8.16-8.24 (m, 2H), 8.41 (d, 1H), 10.49 (s, 1H).

LC-MS (method 2): Rₜ=3.15 min; m/z=412 [M+H]⁺.

### Example 14A

### 2-(2-Chlorophenoxy)-6-(4-fluoro-3-methylphenyl)nicotinaldehyde

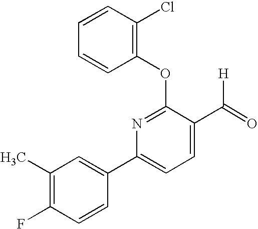

The title compound is prepared and purified analogously to Example 11A. Starting from 95 mg (0.38 mmol) of 2-chloro-6-(4-fluoro-3-methylphenyl)nicotinaldehyde from Example 4A and 49 mg (0.38 mmol) of 2-chlorophenol, 118 mg (91% of theory) of the target compound are obtained.

¹H NMR (400 MHz, DMSO-d₆): δ=2.22 (s, 3H), 7.20 (t, 1H), 7.39 (ddd, 1H), 7.48-7.56 (m, 2H), 7.64-7.70 (m, 2H), 7.82 (dd, 1H), 7.91 (d, 1H), 8.33 (d, 1H), 10.47 (s, 1H).

LC-MS (method 3): Rₜ=4.46 min; m/z=342 [M+H]⁺.

### Example 15A

### 2-(2-Chlorophenoxy)-6-(3-fluoro-4-methylphenyl)nicotinaldehyde

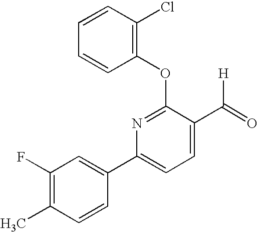

51 mg (0.40 mmol) of 2-chlorophenol and 166 mg (1.20 mmol) of potassium carbonate are added to a solution of 100 mg (0.40 mmol) of 2-chloro-6-(4-fluoro-3-methylphenyl)nicotinaldehyde from Example 5A in 2 ml of DMF. The mixture was left to stir at RT overnight and for a further day, then at 80° C. for 5 h for further completion of the reaction and, after filtration from the solid, purified by preparative HPLC (method 9). This affords 125 mg (91% of theory) of the target compound.

¹H NMR (400 MHz, DMSO-d₆): δ=2.24 (s, 3H), 7.35 (t, 1H), 7.40 (ddd, 1H), 7.48-7.57 (m, 3H), 7.62 (dd, 1H), 7.68 (dd, 1H), 7.95 (d, 1H), 8.34 (d, 1H), 10.47 (s, 1H).

LC-MS (method 6): Rₜ=3.06 min; m/z=342 [M+H]⁺.

### Example 16A

### 2-(2-Chlorophenoxy)-6-(2,3-difluorophenyl)nicotinaldehyde

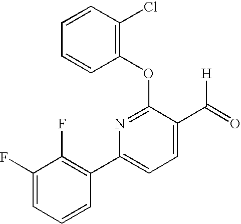

75 mg (0.59 mmol) of 2-chlorophenol and 221 mg (1.60 mmol) of potassium carbonate are added to a solution of 135 mg (0.53 mmol) of 2-chloro-6-(2,3-difluorophenyl)nicotinaldehyde from Example 6A in 4 ml of DMF. Subsequently, the mixture is left to stir at 60° C. overnight. After filtration from the solid, purification by preparative HPLC (method 9) gives 111 mg (60% of theory) of the target compound.

¹H NMR (400 MHz, DMSO-d₆): δ=7.20-7.31 (m, 1H), 7.31-7.42 (m, 2H), 7.42-7.60 (m, 3H), 7.66 (dd, 1H), 7.78 (dd, 1H), 8.42 (d, 1H), 10.50 (s, 1H).

LC-MS (method 3): Rₜ=4.29 min; m/z=346 [M+H]⁺.

### Example 17A

### 2-(2-Chlorophenoxy)-6-(2-chlorophenyl)nicotinaldehyde

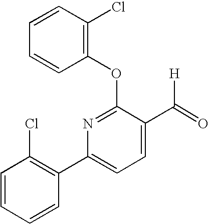

The title compound is prepared and purified analogously to Example 16A. Starting from 125 mg (61% pure, approx. 0.30 mmol) of 2-chloro-6-(2-chlorophenyl)nicotinaldehyde from Example 7A, this affords 85 mg (82% of theory) of the target compound.

¹H NMR (400 MHz, DMSO-d₆): δ=7.31 (td, 1H), 7.37-7.54 (m, 6H), 7.60 (dd, 1H), 7.62 (d, 1H), 8.38 (d, 1H), 10.51 (s, 1H).

LC-MS (method 3): Rₜ=4.27 min; m/z=344 [M+H]⁺.

### Example 18A

### 2-(2-Chlorophenoxy)-6-(2,3-dimethylphenyl)nicotinaldehyde

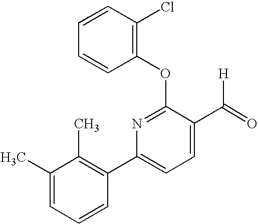

The title compound is prepared and purified analogously to Example 16A. Starting from 140 mg (0.57 mmol) of 2-chloro-6-(2,3-dimethylphenyl)nicotinaldehyde from Example 8A, this affords 158 mg (82% of theory) of the target compound.

¹H NMR (400 MHz, DMSO-d₆): δ=1.97 (s, 3H), 2.21 (s, 3H), 7.10-7.17 (m, 2H), 7.18-7.24 (m, 1H), 7.32 (td, 1H), 7.40-7.49 (m, 3H), 7.60 (dd, 1H), 8.34 (d, 1H), 10.50 (s, 1H).

LC-MS (method 1): Rₜ=3.07 min; m/z=338 [M+H]⁺.

### Example 19A

### 2-(2-Chlorophenoxy)-6-[3-(trifluoromethoxy)phenyl]nicotinaldehyde

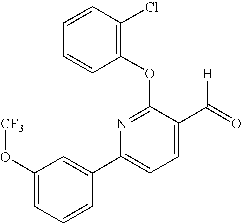

The title compound is prepared and purified analogously to Example 16A. Starting from 110 mg (0.37 mmol) of 2-chloro-6-[3-(trifluoromethoxy)phenyl]nicotinaldehyde from Example 9A, this affords 139 mg (97% of theory) of the target compound.

¹H NMR (400 MHz, DMSO-d₆): δ=7.40 (td, 1H), 7.45 (br. dd, 1H), 7.47-7.57 (m, 2H), 7.59 (t, 1H), 7.67 (dd, 1H), 7.73 (br. t, 1H), 7.95 (br. d, 1H), 8.03 (d, 1H), 8.39 (d, 1H), 10.49 (s, 1H).

LC-MS (method 5): Rₜ=4.38 min; m/z=394 [M+H]⁺.

### Example 20A

### 2-(2-Chlorophenoxy)-6-(2-fluoro-3-methoxyphenyl)nicotinaldehyde

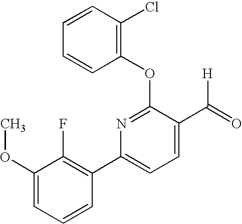

The title compound is prepared and purified analogously to Example 16A. Starting from 100 mg (0.38 mmol) of 2-chloro-6-(2-fluoro-3-methoxyphenyl)nicotinaldehyde from Example 10A, this affords 97 mg (72% of theory) of the target compound.

¹H NMR (400 MHz, DMSO-d₆): δ=3.85 (s, 3H), 7.06 (ddd, 1H), 7.15 (td, 1H), 7.25 (td, 1H), 7.36 (td, 1H), 7.44-7.54 (m, 2H), 7.65 (dd, 1H), 7.74 (dd, 1H), 8.38 (d, 1H), 10.49 (s, 1H).

LC-MS (method 5): Rₜ=4.03 min; m/z=358 [M+H]⁺.

### Example 21A

### 2-Chloro-6-(3-fluoro-4-methylphenyl)-4-(trifluoromethyl)nicotinamide

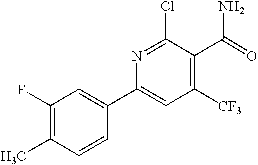

154 mg (1.00 mmol) of 3-fluoro-4-methylphenylboronic acid and 3.00 ml (6.00 mmol) of a 2 M aqueous potassium carbonate solution are added to 259 mg (1.00 mmol) of 2,6-dichloro-4-(trifluoromethyl)nicotinamide, dissolved in 3.5 ml of DMF. After stirring for 10 min, 140 mg (0.20 mmol) of bis(triphenylphosphine)palladium(II) chloride and 30.4 mg (0.10 mmol) of tri-2-tolylphosphine are added and the reaction mixture is stirred at RT overnight. For workup, the reaction mixture is partitioned between ethyl acetate and water, and acidified to pH 3.5 with 1N hydrochloric acid, the organic phase is removed, the aqueous phase is extracted once more with ethyl acetate, and the combined organic phases are dried over magnesium sulfate and concentrated. The remaining crude product is purified by preparative HPLC (method 8). 200 mg (60% of theory) of the target compound are thus obtained.

¹H NMR (400 MHz, DMSO-d₆): δ=2.32 (s, 3H), 7.48 (t, 1H), 7.94-7.82 (m, 2H), 8.06 (br. s, 1H), 8.21 (br. s, 1H), 8.39 (s, 1H).

LC-MS (method 2): Rₜ=2.19 min; m/z=333 [M+H]⁺.

### Example 22A

### 2-(2-Chlorophenoxy)-6-(3-fluoro-4-methylphenyl)-4-(trifluoromethyl)nicotinamide

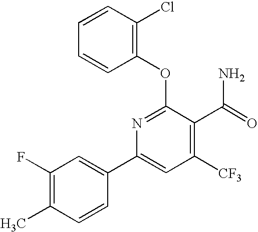

75 mg (0.59 mmol) of 2-chlorophenol and 243 mg (1.76 mmol) of potassium carbonate are added with stirring to 195 mg (0.59 mmol) of 2-chloro-6-(3-fluoro-4-methylphenyl)-4-(trifluoromethyl)-nicotinamide from Example 21A, dissolved in 5.0 ml of DMF. The mixture is stirred first at RT overnight then at 60° C. for a further two days. For workup and purification, the liquid phase of the mixture is separated directly by means of preparative HPLC (method 8). This affords 174 mg (70% of theory) of the target compound.

LC-MS (method 3): Rₜ=3.89 min; m/z=425 [M+H]⁺.

### Example 23A

### Methyl 2-chloro-6-(3-fluoro-4-methylphenyl)nicotinate

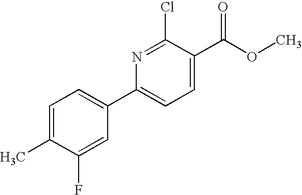

Under an argon atmosphere, 2.33 ml (1.17 mmol) of a 0.5 M solution of 3-fluoro-4-methylphenylzinc iodide in THF and 56 mg (0.049 mmol) of tetrakis(triphenylphosphine)-palladium(0) are added to a solution of 200 mg (0.97 mmol) of methyl 2,6-dichloronicotinate in 3.0 ml of DMF, and the mixture is left to stir at RT overnight. For workup, the mixture is stirred with 30 ml of water and 15 ml of ethyl acetate and filtered with suction through 2 g of Celite. The organic phase is removed and concentrated, and the remaining residue is purified by preparative HPLC (method 9). This affords 113 mg (42% of theory) of the target compound.

¹H NMR (400 MHz, DMSO-d₆): δ=2.31 (d, 3H), 3.90 (s, 3H), 7.47 (t, 1H), 7.86-7.94 (m, 2H), 8.17 (d, 1H), 8.34 (d, 1H).

LC-MS (method 5): Rₜ=3.90 min; m/z=280 [M+H]⁺.

### Example 24A

### Methyl 2-(2-chloro-5-methoxyphenoxy)-6-(3-fluoro-4-methylphenyl)nicotinate

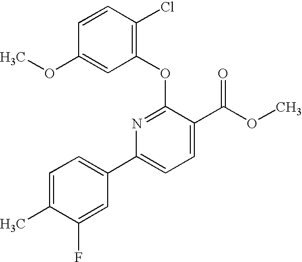

31 mg (0.20 mmol) of 2-chloro-5-methoxyphenol and 74 mg (0.54 mmol) of potassium carbonate are added to a solution of 50 mg (0.18 mmol) of methyl 2-chloro-6-(3-fluoro-4-methylphenyl)-nicotinate from Example 23A in 2.0 ml of DMF, and the mixture is first left to stir at 60° C. overnight. A further 74 mg (0.54 mmol) of potassium carbonate and about 300 mg of molecular sieve (4 Å) are added and the mixture is stirred over one night each at 60° C., then at 80° C. and finally at 100° C. For workup and purification, the mixture is filtered and the filtrate is separated by means of preparative HPLC (method 9). This affords 52 mg (72% of theory) of the target compound.

¹H NMR (400 MHz, DMSO-d₆): δ=2.24 (s, 3H), 3.78 (s, 3H), 3.90 (s, 3H), 6.94 (dd, 1H), 7.03 (d, 1H), 7.35 (t, 1H), 7.51 (d, 1H), 7.53 (d, 1H), 7.60 (d, 1H), 7.87 (d, 1H), 8.39 (d, 1H).

LC-MS (method 3): Rₜ=4.41 min; m/z=402 [M+H]⁺.

### Example 25A

### 2-(2-Chlorophenoxy)-6-phenylnicotinonitrile

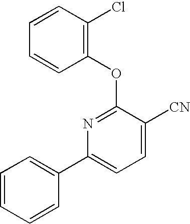

Under an argon atmosphere, 773 mg (5.59 mmol) of potassium carbonate are added to a solution of 600 mg (2.80 mmol) of 2-chloro-6-phenylnicotinonitrile and 395 mg (3.08 mmol) of 2-chlorophenol in 12 ml of DMF. The mixture is left to stir first at RT overnight and then at 60° C. for a further day. Direct purification by preparative HPLC gives 730 mg (85% of theory) of the target compound.

¹H NMR (400 MHz, DMSO-d₆): δ=7.37-7.48 (m, 4H), 7.48-7.58 (m, 2H), 7.69 (d, 1H), 7.82 (d, 2H), 7.95 (d, 1H), 8.53 (d, 1H).

LC-MS (method 4): Rₜ=2.90 min; m/z=307 [M+H]⁺.

### Example 26A

### 2-(2-Chlorophenoxy)-6-(4-fluorophenyl)nicotinonitrile

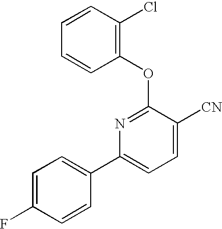

Under an argon atmosphere, 152 mg (1.18 mmol) of 2-chlorophenol and 297 mg (2.15 mmol) of potassium carbonate are added to a solution of 250 mg (1.08 mmol) of 2-chloro-6-(4-fluorophenyl)nicotinonitrile in 5 ml of DMF. The mixture is left to stir at 60° C. overnight and, after filtering from the solid, purified by preparative HPLC (method 9). This affords 325 mg (93% of theory) of the target compound.

¹H NMR (400 MHz, DMSO-d₆): δ=7.25-7.33 (m, 2H), 7.41 (td, 1H), 7.48-7.57 (m, 2H), 7.68 (dd, 1H), 7.84-7.92 (m, 2H), 7.95 (d, 1H), 8.53 (d, 1H).

LC-MS (method 2): Rₜ=2.76 min; m/z=325 [M+H]⁺.

### Example 27A

### 2-(2-Chlorophenoxy)-6-(4-chlorophenyl)nicotinonitrile

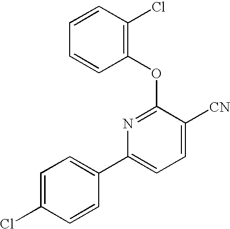

Under an argon atmosphere, 142 mg (1.10 mmol) of 2-chlorophenol and 277 mg (2.01 mmol) of potassium carbonate are added to a solution of 250 mg (1.00 mmol) of 2-chloro-6-(4-chlorophenyl)nicotinonitrile in 5 ml of DMF. The mixture is left to stir at 60° C. overnight, then at 80° C. for 4 h for further completion of the reaction and, after filtration from the solid, purified by preparative HPLC (method 9). This affords 320 mg (93% of theory) of the target compound.

¹H NMR (400 MHz, DMSO-d₆): δ=7.41 (td, 1H), 7.48-7.57 (m, 4H), 7.68 (dd, 1H), 7.83 (d, 2H), 7.97 (d, 1H), 8.55 (d, 1H).

LC-MS (method 4): Rₜ=3.09 min; m/z=341 [M+H]⁺.

### Example 28A

### 6,6′-Dichloro-2,3′-bipyridine-5-carboxaldehyde

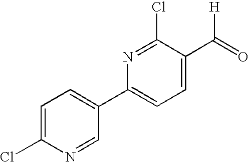

The title compound is prepared and purified initially analogously to Example 1A. After a second preparative HPLC separation (method 9) followed by a silica gel chromatography (eluent: 80:1 dichloromethane/methanol), starting from 200 mg (1.14 mmol) of 2,6-dichloropyridine-3-carboxaldehyde, 179 mg (68% of theory) of the target compound are obtained, which are reacted further without complete purification.

LC-MS (method 1): Rₜ=2.36 min; m/z=253 [M+H]⁺.

### Example 29A 6′-Chloro-6-(2-chlorophenoxy)-2,3′-bipyridine-5-carboxaldehyde

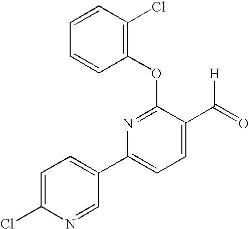

86 mg (0.67 mmol) of 2-chlorophenol and 278 mg (2.02 mmol) of potassium carbonate are added to 170 mg (0.67 mmol) of 6,6′-dichloro-2,3′-bipyridine-5-carboxaldehyde from Example 28A dissolved in 5.00 ml of DMF. The mixture is stirred overnight and left to stand at RT for three further days. For workup and purification, the filtrate is filtered from the solid and separated by preparative HPLC (method 9). This affords 158 mg (67% of theory) of the target compound.

¹H NMR (400 MHz, DMSO-d₆): δ=7.38 (td, 1H), 7.48-7.58 (m, 2H), 7.63 (d, 1H), 7.68 (dd, 1H), 8.05 (d, 1H), 8.21 (dd, 1H), 8.41 (d, 1H), 8.82 (d, 1H), 10.49 (s, 1H).

LC-MS (method 6): Rₜ=2.75 min; m/z=345 [M+H]⁺.

### Example 30A

### tert-Butyl 2,6-dichloronicotinate

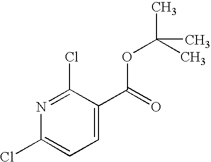

10.0 g (52.1 mmol) of 2,6-dichloronicotinic acid [D. Laeckmann et al., Bioorg. Med. Chem. 10, 1793-1804 (2002)] are suspended in 100 ml of tert-butanol and admixed with ice cooling with 62.6 g (312.5 mmol) of O-tert-butyl N,N′-diisopropylimidocarbamate [K. R. West et al., Org. Lett. 13, 2615-2618 (2005)]. The resulting clear solution is stirred at room temperature overnight. The resulting precipitate is then removed by means of filtration. The mother liquor is concentrated on a rotary evaporator and the residue is taken up in ethyl acetate. The mixture is washed with water and the organic phase is dried over sodium sulfate. The solvent is removed under reduced pressure and the crude product is purified by means of column chromatography on silica gel (eluent: 7:3 cyclohexane/ethyl acetate). This affords 9.67 g (73% of theory) of the target compound.

¹H NMR (400 MHz, DMSO-d₆): δ=1.56 (s, 9H), 7.70 (d, 1H), 8.26 (d, 1H).

LC-MS (method 11): Rₜ=2.41 min; m/z=248 [M+H]⁺.

### Example 31A

### tert-Butyl 2-chloro-6-(3,5-difluorophenyl)nicotinate

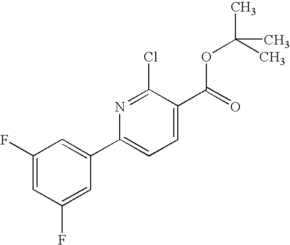

5.00 g (20.1 mmol) of the compound from Example 30A are taken up in 100 ml 1,2-dimethoxy-ethane and admixed with 3.18 g (20.1 mmol) of 3,5-difluorophenylboronic acid and 16.7 g (120.9 mmol) of potassium carbonate. After stirring at room temperature for 10 minutes, 707 mg (1.01 mmol) of bis(triphenylphosphine)palladium(II) chloride and 613 mg (2.02 mmol) of tri-2-tolylphosphine are added. The reaction mixture is stirred at 60° C. overnight. Thereafter, 200 ml of ethyl acetate are added and the mixture is washed twice with 100 ml each time of saturated sodium chloride solution. The organic phase is dried and concentrated. The residue is prepurified by column chromatography on silica gel with cyclohexane/ethyl acetate (10:1) as the eluent. The end purification is performed by means of preparative HPLC (column: Chromatorex C18; eluent: acetonitrile/water 9:1). This affords 1.78 g (27% of theory) of the target compound.

¹H NMR (400 MHz, DMSO-d₆): δ=1.58 (s, 9H), 7.43 (tt, 1H), 7.84 (m_{z}, 2H), 8.21 (d, 1H), 8.30 (d, 1H).

LC-MS (method 1): Rₜ=3.28 min; m/z=326 [M+H]⁺.

### Example 32A

### 4-Chloro-3-hydroxybenzonitrile

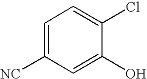

500 mg (2.41 mmol) of 5-bromo-2-chlorophenol, 139 mg (0.121 mmol) of tetrakis(tri-phenylphosphine)palladium(0) and 209 mg (1.78 mmol) of zinc cyanide are taken up in 5 ml of DMF. Subsequently, the mixture is converted in a single mode microwave (Emrys Optimizer) at 220° C. for 5 min. The crude product is separated directly by means of preparative HPLC (eluent: acetonitrile/water with 0.1% formic acid, gradient 20:80→95:5). This affords 240 mg (65% of theory) of the target compound.

LC-MS (method 12): Rₜ=1.30 min; MS (EIneg): m/z=152 [M−H]⁻.

### Example 33A

### tert-Butyl 2-(2-chloro-5-cyanophenoxy)-6-(3,5-difluorophenyl)nicotinate

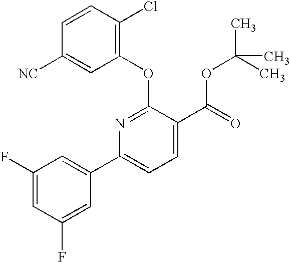

100.0 mg (0.307 mmol) of the compound from Example 31A, 47.1 mg (0.307 mmol) of the compound from Example 32A and 84.9 mg (0.614 mmol) of potassium carbonate are reacted in 1.8 ml of DMF in a shaker at 100° C. over 24 h. Subsequently, the salts are removed by filtration and the crude product is purified by means of preparative HPLC (eluent: acetonitrile/water with 0.1% formic acid, gradient 20:80→95:5). This affords 97 mg (50% of theory) of the target compound in 70% purity.

LC-MS (method 12): Rₜ=2.77 min; m/z=443 [M+H]⁺.

### Example 34A

### tert-Butyl 2,6-dichloro-4-(trifluoromethyl)nicotinate

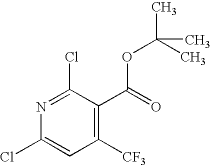

10.0 g (38.5 mmol) of 2,6-dichloro-4-(trifluoromethyl)nicotinic acid [Y. Tsuzuki et al., J. Med. Chem. 47, 2097-2109 (2004)] are suspended in 70 ml of tert-butanol and admixed with ice cooling with 46.2 g (200.3 mmol) of O-tert-butyl N,N′-diisopropylimidocarbamate [K. R. West et al., Org. Lett. 13, 2615-2618 (2005)]. The resulting clear solution is stirred at room temperature overnight. The resulting precipitate is then removed by filtration. The mother liquor is concentrated on a rotary evaporator and the residue is taken up in ethyl acetate. The mixture is washed with water and the organic phase is dried over sodium sulfate. The solvent is removed under reduced pressure and the crude product is purified by means of column chromatography on silica gel (eluent: 7:3 cyclohexane/ethyl acetate). This affords 8.95 g (73% of theory) of the target compound.

¹H NMR (400 MHz, DMSO-d₆): δ=1.56 (s, 9H), 8.22 (s, 1H).

LC-MS (method 11): Rₜ=2.73 min; m/z=316 [M+H]⁺.

### Example 35A

### tert-Butyl 2-chloro-6-(3,5-difluorophenyl)-4-(trifluoromethyl)nicotinate

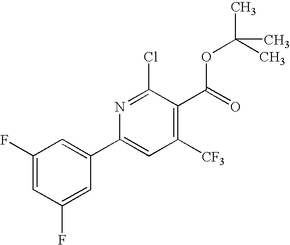

4.00 g (12.6 mmol) of the compound from Example 34A are taken up in 100 ml of 1,4-dioxane and admixed with 2.00 g (12.6 mmol) of 3,5-difluorophenylboronic acid and 10.5 g (75.9 mmol) of potassium carbonate (as solution in 37 ml of water). After stirring at room temperature for 10 minutes, 888 mg (1.26 mmol) of bis(triphenylphosphine)palladium(II) chloride and 385 mg (1.26 mmol) of tri-2-tolylphosphine are added. The reaction mixture is stirred at 60° C. overnight. Thereafter, 200 ml of ethyl acetate are added and the mixture is washed with 100 ml of water. The organic phase is dried and concentrated. The residue is recrystallized from ethanol. This affords 2.29 g (46% of theory) of the target compound.

¹H NMR (400 MHz, DMSO-d₆): δ=1.58 (s, 9H), 7.50 (tt, 1H), 7.95 (m_{z}, 2H), 8.57 (s, 1H).

LC-MS (method 12): Rₜ=2.89 min; m/z=394 [M+H]⁺.

### Example 36A

### tert-Butyl 2-(2,5-difluorophenoxy)-6-(3,5-difluorophenyl)-4-(trifluoromethyl)nicotinate

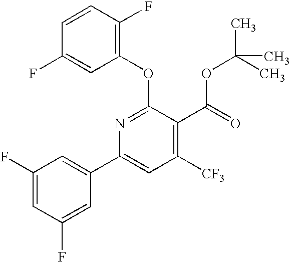

100.0 mg (0.254 mmol) of the compound from Example 35A, 33.0 mg (0.254 mmol) of 2,5-difluorophenol and 70.0 mg (0.508 mmol) of potassium carbonate are reacted in 2 ml of DMF in a shaker at 70° C. over 14 h. Subsequently, the mixture is purified directly by means of preparative HPLC (eluent: acetonitrile/water with 0.1% formic acid, gradient 20:80→95:5). This affords 70 mg (57% of theory) of the target compound.

¹H NMR (400 MHz, DMSO-d₆): δ=1.56 (s, 9H), 7.29 (m_{z}, 1H), 7.41 (tt, 1H), 7.51-7.60 (m, 2H), 7.66 (m_{z}, 2H), 8.33 (s, 1H).

LC-MS (method 1): Rₜ=3.47 min; m/z=488 [M+H]⁺.

### Example 37A

### tert-Butyl 2-(4-bromo-2-fluorophenoxy)-6-(3,5-difluorophenyl)-4-(trifluoromethyl)nicotinate

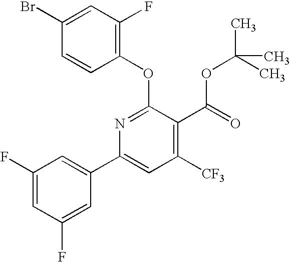

100.0 mg (0.254 mmol) of the compound from Example 35A, 49.0 mg (0.254 mmol) of 4-bromo-2-fluorophenol and 70.0 mg (0.508 mmol) of potassium carbonate are reacted in 2 ml of DMF in a shaker at 70° C. over 14 h. Subsequently, the mixture is purified directly by means of preparative HPLC (eluent: acetonitrile/water with 0.1% formic acid, gradient 20:80→95:5). This affords 60 mg (43% of theory) of the target compound.

¹H NMR (400 MHz, DMSO-d₆): δ=1.57 (s, 9H), 7.41 (tt, 1H), 7.48-7.60 (m, 2H), 7.67 (m_{z}, 2H), 7.87 (dd, 1H), 8.31 (s, 1H).

LC-MS (method 11): Rₜ=3.33 min; m/z=549 [M+H]⁺.

### Example 38A

### 2-(2-Chloro-5-trifluoromethylphenoxy)-6-(2,3-difluorophenyl)nicotinaldehyde

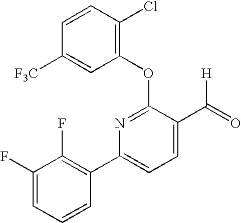

The title compound is prepared and purified analogously to Example 16A. Starting from 50 mg (0.20 mmol) of 2-chloro-6-(2,3-difluorophenyl)nicotinaldehyde from Example 6A, 72 mg (88% of theory) of the target compound are obtained.

¹H NMR (400 MHz, DMSO-d₆): δ=7.26 (tdd, 1H), 7.35 (ddt, 1H), 7.54 (dddd, 1H), 7.75 (dd, 1H), 7.82 (dd, 1H), 7.93 (d, 1H), 8.07 (d, 1H), 8.45 (s, 1H), 10.50 (s, 1H).

LC-MS (method 3): Rₜ=4.56 min; m/z=414 [M+H]⁺.

### Example 39A

### 2-(2-Chloro-4-trifluoromethoxyphenoxy)-6-(2,3-difluorophenyl)nicotinaldehyde

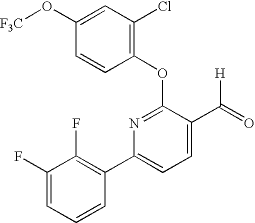

The title compound is prepared and purified analogously to Example 16A. Starting from 50 mg (0.20 mmol) of 2-chloro-6-(2,3-difluorophenyl)nicotinaldehyde from Example 6A, 61 mg (72% of theory) of the target compound are obtained.

¹H NMR (400 MHz, DMSO-d₆): δ=7.25 (tdd, 1H), 7.36 (ddt, 1H), 7.50-7.60 (m, 2H), 7.69 (d, 1H), 7.81 (dd, 1H), 7.84 (d, 1H), 8.44 (d, 1H), 10.48 (s, 1H).

LC-MS (method 3): Rₜ=4.62 min; m/z=430 [M+H]⁺.

### Example 40A

### 2-(2-Chloro-4-methoxyphenoxy)-6-(2,3-difluorophenyl)nicotinaldehyde

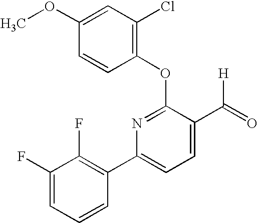

The title compound is prepared and purified analogously to Example 16A. Starting from 50 mg (0.20 mmol) of 2-chloro-6-(2,3-difluorophenyl)nicotinaldehyde from Example 6A, 41 mg (54% of theory) of the target compound are obtained.

¹H NMR (400 MHz, DMSO-d₆): δ=3.78 (s, 3H), 6.94 (dd, 1H), 7.17 (d, 1H), 7.28 (tdd, 1H), 7.39 (ddt, 1H), 7.49-7.59 (m, 1H), 7.54 (d, 1H), 7.78 (dd, 1H), 8.41 (d, 1H), 10.49 (s, 1H).

LC-MS (method 5): Rₜ=4.13 min; m/z=376 [M+H]⁺.

### Example 41A

### 2-(2-Fluoro-5-methylphenoxy)-6-(2,3-difluorophenyl)nicotinaldehyde

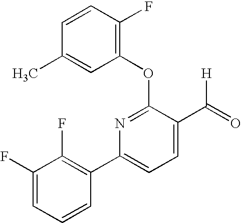

The title compound is prepared and purified analogously to Example 16A. Starting from 50 mg (0.20 mmol) of 2-chloro-6-(2,3-difluorophenyl)nicotinaldehyde from Example 6A, 35 mg (52% of theory) of the target compound are obtained.

¹H NMR (400 MHz, DMSO-d₆): δ=2.32 (s, 3H), 7.12-7.18 (m, 1H), 7.24-7.35 (m, 3H), 7.39 (ddt, 1H), 7.55 (dddd, 1H), 7.78 (dd, 1H), 8.40 (d, 1H), 10.46 (s, 1H).

LC-MS (method 3): Rₜ=4.34 min; m/z=344 [M+H]⁺.

### Example 42A

### 2-(2-Methoxyphenoxy)-6-(2,3-difluorophenyl)nicotinaldehyde

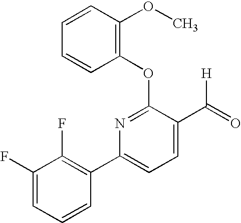

The title compound is prepared and purified analogously to Example 16A. Starting from 50 mg (0.20 mmol) of 2-chloro-6-(2,3-difluorophenyl)nicotinaldehyde from Example 6A, 24 mg (36% of theory) of the target compound are obtained.

¹H NMR (400 MHz, DMSO-d₆): δ=3.71 (s, 3H), 7.03 (td, 1H), 7.17-7.36 (m, 5H), 7.52 (dddd, 1H), 7.71 (dd, 1H), 8.35 (d, 1H), 10.48 (s, 1H).

LC-MS (method 5): Rₜ=3.91 min; m/z=342 [M+H]⁺.

### Example 43A

### 2-(2-Fluoro-5-trifluoromethylphenoxy)-6-(2,3-difluorophenyl)nicotinaldehyde

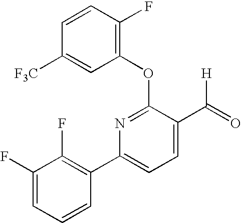

The title compound is prepared and purified analogously to Example 16A. Starting from 50 mg (0.20 mmol) of 2-chloro-6-(2,3-difluorophenyl)nicotinaldehyde from Example 6A, 71 mg (91% of theory) of the target compound are obtained.

¹H NMR (400 MHz, DMSO-d₆): δ=7.27 (td, 1H), 7.38 (ddt, 1H), 7.55 (dddd, 1H), 7.72 (t, 1H), 7.76-7.86 (m, 1H), 7.83 (d, 1H), 8.05 (dd, 1H), 8.44 (d, 1H), 10.47 (s, 1H).

LC-MS (method 5): Rₜ=4.22 min; m/z=398 [M+H]⁺.

### Example 44A

### 2-(2-Trifluoromethoxyphenoxy)-6-(2,3-difluorophenyl)nicotinaldehyde

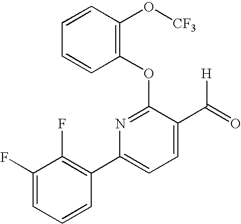

The title compound is prepared and purified analogously to Example 16A. Starting from 50 mg (0.20 mmol) of 2-chloro-6-(2,3-difluorophenyl)nicotinaldehyde from Example 6A, 74 mg (95% of theory) of the target compound are obtained.

¹H NMR (400 MHz, DMSO-d₆): δ=7.26 (tdd, 1H), 7.36 (ddt, 1H), 7.45 (td, 1H), 7.49-7.62 (m, 4H), 7.81 (dd, 1H), 8.43 (d, 1H), 10.45 (s, 1H).

LC-MS (method 5): Rₜ=4.20 min; m/z=396 [M+H]⁺.

### Example 45A

### 2-(2-Fluorophenoxy)-6-(2,3-difluorophenyl)nicotinaldehyde

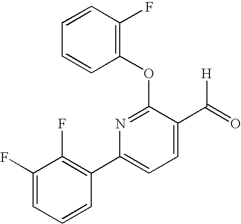

The title compound is prepared and purified analogously to Example 16A. Starting from 50 mg (0.20 mmol) of 2-chloro-6-(2,3-difluorophenyl)nicotinaldehyde from Example 6A, 35 mg (54% of theory) of the target compound are obtained.

¹H NMR (400 MHz, DMSO-d₆): δ=7.22-7.59 (m, 7H), 7.79 (dd, 1H), 8.41 (d, 1H), 10.47 (s, 1H).

LC-MS (method 5): Rₜ=3.97 min; m/z=330 [M+H]⁺.

### Example 46A

### 2-(2-Chloro-5-methylphenoxy)-6-(2,3-difluorophenyl)nicotinaldehyde

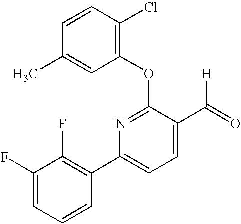

The title compound is prepared and purified analogously to Example 16A. Starting from 50 mg (0.20 mmol) of 2-chloro-6-(2,3-difluorophenyl)nicotinaldehyde from Example 6A, 29 mg (41% of theory) of the target compound are obtained.

¹H NMR (400 MHz, DMSO-d₆): δ=2.34 (s, 3H), 7.17 (dd, 1H), 7.23-7.31 (m, 1H), 7.32-7.39 (m, 2H), 7.49-7.59 (m, 1H), 7.52 (d, 1H), 7.77 (dd, 1H), 8.41 (d, 1H), 10.48 (s, 1H).

LC-MS (method 5): Rₜ=4.30 min; m/z=360 [M+H]⁺.

### Example 47A

### 2-(2-Methylphenoxy)-6-(2,3-difluorophenyl)nicotinaldehyde

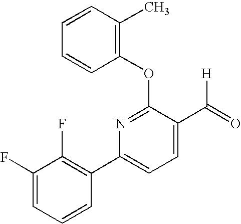

The title compound is prepared and purified analogously to Example 16A. Starting from 50 mg (0.20 mmol) of 2-chloro-6-(2,3-difluorophenyl)nicotinaldehyde from Example 6A, 31 mg (48% of theory) of the target compound are obtained.

¹H NMR (400 MHz, DMSO-d₆): δ=2.18 (s, 3H), 7.18-7.33 (m, 4H), 7.33-7.39 (m, 2H), 7.53 (dddd, 1H), 7.73 (dd, 1H), 8.38 (d, 1H), 10.50 (s, 1H).

LC-MS (method 1): Rₜ=3.09 min; m/z=326 [M+H]⁺.

### Example 48A

### 2-(5-Chloro-2-methylphenoxy)-6-(2,3-difluorophenyl)nicotinaldehyde

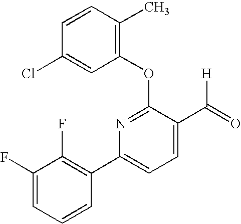

The title compound is prepared and purified analogously to Example 16A. Starting from 50 mg (0.20 mmol) of 2-chloro-6-(2,3-difluorophenyl)nicotinaldehyde from Example 6A, 22 mg (31% of theory) of the target compound are obtained.

¹H NMR (400 MHz, DMSO-d₆): δ=2.17 (s, 3H), 7.24-7.32 (m, 2H), 7.38 (ddt, 1H), 7.40 (d, 1H), 7.45 (d, 1H), 7.54 (dddd, 1H), 7.76 (dd, 1H), 8.39 (d, 1H), 10.47 (s, 1H).

LC-MS (method 1): Rₜ=3.23 min; m/z=360 [M+H]⁺.

### Example 49A

### 2-(2-Trifluoromethylphenoxy)-6-(2,3-difluorophenyl)nicotinaldehyde

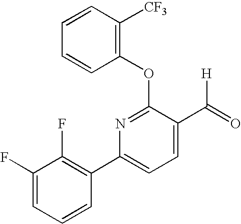

The title compound is prepared and purified analogously to Example 16A. Starting from 50 mg (0.20 mmol) of 2-chloro-6-(2,3-difluorophenyl)nicotinaldehyde from Example 6A, 64 mg (87% of theory) of the target compound are obtained.

¹H NMR (400 MHz, DMSO-d₆): δ=7.27 (dddd, 1H), 7.39 (ddt, 1H), 7.49-7.59 (m, 2H), 7.65 (d, 1H), 7.78-7.85 (m, 2H), 7.87 (br. d, 1H), 8.43 (d, 1H), 10.44 (s, 1H).

LC-MS (method 1): Rₜ=3.11 min; m/z=380 [M+H]⁺.

### Example 50A

### 2-(2,5-Difluorophenoxy)-6-(2,3-difluorophenyl)nicotinaldehyde

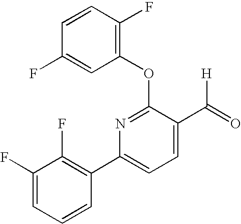

The title compound is prepared and purified analogously to Example 16A. Starting from 50 mg (0.20 mmol) of 2-chloro-6-(2,3-difluorophenyl)nicotinaldehyde from Example 6A, 58 mg (85% of theory) of the target compound are obtained.

¹H NMR (400 MHz, DMSO-d₆): δ=7.21-7.33 (m, 2H), 7.40 (ddt, 1H), 7.48-7.60 (m, 3H), 7.82 (dd, 1H), 8.43 (d, 1H), 10.45 (s, 1H).

LC-MS (method 1): Rₜ=3.00 min; m/z=348 [M+H]⁺.

### Example 51A

### 2-(2-Chloro-5-methoxyphenoxy)-6-(2,3-difluorophenyl)nicotinaldehyde

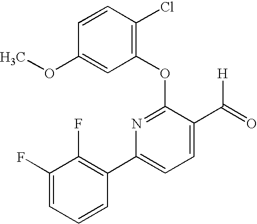

The title compound is prepared and purified analogously to Example 16A. Starting from 50 mg (0.20 mmol) of 2-chloro-6-(2,3-difluorophenyl)nicotinaldehyde from Example 6A, 40 mg (54% of theory) of the target compound are obtained.

¹H NMR (400 MHz, DMSO-d₆): δ=3.82 (s, 3H), 7.03 (dd, 1H), 7.23 (d, 1H), 7.28 (tdd, 1H), 7.38 (ddt, 1H), 7.44 (d, 1H), 7.55 (dddd, 1H), 7.78 (dd, 1H), 8.39 (d, 1H), 10.49 (s, 1H).

LC-MS (method 1): Rₜ=3.11 min; m/z=376 [M+H]⁺.

### Example 52A

### Methyl 2-(2-chloro-4-methoxyphenoxy)-6-(3-fluoro-4-methylphenyl)nicotinate

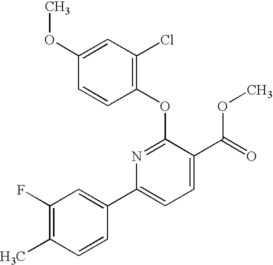

29 mg (0.19 mmol) of 2-chloro-4-methoxyphenol and 70 mg (0.50 mmol) of potassium carbonate are added to a solution of 47 mg (0.17 mmol) of methyl 2-chloro-6-(3-fluoro-4-methylphenyl)-nicotinate from Example 23A in 2.5 ml of DMF, and the mixture is left to stir at 100° C. overnight. For workup and purification, the mixture is filtered and the filtrate is separated by means of preparative HPLC (method 9). This affords 63 mg (93% of theory) of the target compound.

¹H NMR (400 MHz, DMSO-d₆): δ=2.23 (s, 3H), 3.83 (s, 3H), 3.90 (s, 3H), 7.03 (dd, 1H), 7.23 (d, 1H), 7.33 (d, 1H), 7.35 (t, 1H), 7.50 (dd, 1H), 7.59 (dd, 1H), 7.84 (d, 1H), 8.37 (d, 1H).

LC-MS (method 1): Rₜ=3.11 min; m/z=402 [M+H]⁺.

### Example 53A

### tert-Butyl 2-chloro-6-(2-fluoro-3-methoxyphenyl)nicotinate

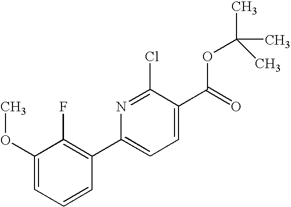

448 mg (2.64 mmol) of 2-fluoro-3-methoxyphenylboronic acid and then 7.9 ml of a 2 M aqueous potassium carbonate solution are added with stirring to a solution of 654 mg (2.64 mmol) of tert-butyl 2,6-dichloronicotinate (Example 30A) in 13 ml of dioxane. After 10 min, 185 mg (0.26 mmol) of bis(triphenylphosphine)palladium(II) chloride and 80 mg (0.26 mmol) of tri-2-tolylphosphine are added, then the reaction mixture is stirred at 60° C. for 5.5 h and subsequently left to stand at RT overnight. For workup, the mixture is taken up with 50 ml of ethyl acetate and 20 ml of saturated aqueous sodium chloride solution, and the organic phase removed is washed once more with saturated aqueous sodium chloride solution, dried over magnesium sulfate and concentrated under reduced pressure. Purification is effected by chromatography on about 100 ml of silica gel with ethyl acetate/cyclohexane (1:5) as the eluent. Isolation of the product fractions and removal of the solvents under reduced pressure affords 638 mg (72% of theory) of the target compound.

¹H NMR (400 MHz, DMSO-d₆): δ=1.58 (s, 9H), 3.90 (s, 3H), 7.27-7.37 (m, 2H), 7.44 (ddd, 1H), 7.90 (dd, 1H), 8.29 (d, 1H).

LC-MS (method 5): Rₜ=4.21 min; m/z=338 [M+H]⁺.

### Example 54A

### tert-Butyl 2-(2,5-difluorophenoxy)-6-(2-fluoro-3-methoxyphenyl)nicotinate

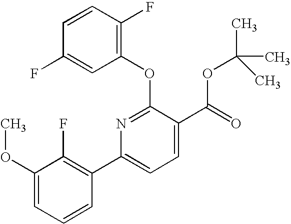

69 mg (0.53 mmol) of 2,5-difluorophenol and 184 mg (1.33 mmol) of potassium carbonate are added to a solution of 150 mg (0.44 mmol) of tert-butyl 2-chloro-6-(2-fluoro-3-methoxyphenyl)-nicotinate from Example 54A in 5 ml of DMF. The mixture is left to stir at 60° C. for three days and, after filtration from the solid, purified by preparative HPLC (method 8). This affords 68 mg (35% of theory) of the target compound.

¹H NMR (400 MHz, DMSO-d₆): δ=1.56 (s, 9H), 3.86 (s, 3H), 7.07 (ddd, 1H), 7.12-7.28 (m, 3H), 7.40 (ddd, 1H), 7.46 (td, 1H), 7.68 (dd, 1H), 8.36 (d, 1H).

LC-MS (method 1): Rₜ=3.26 min; m/z=432 [M+H]⁺.

### Example 55A

### Methyl 2-chloro-5-fluoro-6-(3-fluoro-4-methylphenyl)nicotinate

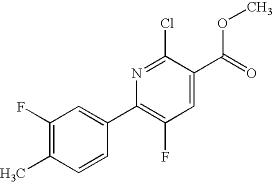

The title compound is prepared and purified analogously to Example 23A. Starting from 200 mg (0.76 mmol) of methyl 2,6-dichloro-5-fluoronicotinate, 85 mg (38% of theory) of the target compound are thus obtained.

¹H NMR (400 MHz, DMSO-d₆): δ=2.33 (d, 3H), 3.92 (s, 3H), 7.50 (t, 1H), 7.70 (d, 1H), 7.74 (br. d, 1H), 8.36 (d, 1H).

LC-MS (method 3): Rₜ=4.24 min; m/z=298 [M+H]⁺.

### Example 56A

### tert-Butyl 2,6-dichloro-5-fluoronicotinate

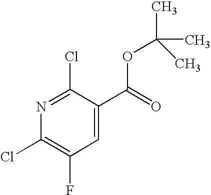

5.72 g (28.6 mmol) of O-tert-butyl N,N′-diisopropylimidocarbamate are added to 1.00 g (4.76 mmol) of 2,6-dichloro-5-fluoronicotinic acid suspended in 15 ml of tert-butanol, and the mixture is stirred at RT overnight. The mixture is then filtered from the precipitate formed, the mother liquor is concentrated, the residue is stirred with 20 ml of ethyl acetate and 20 ml of water, the organic phase is isolated, the aqueous phase is washed once more with 20 ml of ethyl acetate, and the combined organic phases are dried over sodium sulfate and, after filtration, concentrated. The residue is purified on silica gel with cyclohexane/ethyl acetate (20:1) as the eluent. This affords 1.18 g (93% of theory) of the target compound.

¹H NMR (400 MHz, DMSO-d₆): δ=1.56 (s, 9H), 8.42 (d, 1H).

LC-MS (method 1): Rₜ=2.88 min; m/z=210 [M+H—C₄H₈]⁺.

### Example 57A

### tert-Butyl 2-chloro-5-fluoro-6-(3-trifluoromethylphenyl)nicotinate

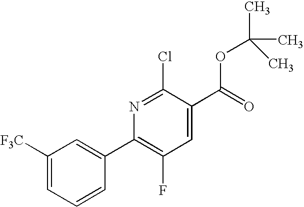

107 mg (2.64 mmol) of 3-trifluoromethylphenylboronic acid and then 1.7 ml of a 2 M aqueous potassium carbonate solution are added with stirring to a solution of 150 mg (0.56 mmol) of tert-butyl 2,6-dichloro-5-fluoronicotinate from Example 56A in 3 ml of dioxane. After 10 min, 40 mg (0.056 mmol) of bis(triphenylphosphine)palladium(II) chloride and 17 mg (0.056 mmol) of tri-2-tolylphosphine are added, and then the reaction mixture is stirred at 60° C. overnight. After purification by preparative HPLC (method 13), 183 mg (86% of theory) of the target compound are thus obtained.

¹H NMR (400 MHz, DMSO-d₆): δ=1.59 (s, 9H), 7.83 (br. t, 1H), 7.95 (br. d, 1H), 8.21 (br. s, 1H), 8.24 (br. d, 1H), 8.37 (d, 1H).

LC-MS (method 5): Rₜ=4.60 min; m/z=376 [M+H]⁺.

### Example 58A

### tert-Butyl 2-(2-chlorophenoxy)-5-fluoro-6-(3-trifluoromethylphenyl)nicotinate

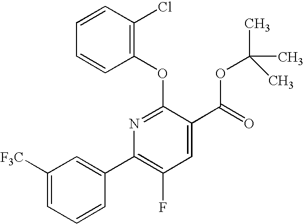

An argon-filled reaction flask is initially charged with 175 mg (0.47 mmol) of tert-butyl 2-chloro-5-fluoro-6-(3-trifluoromethylphenyl)nicotinate from Example 57A, 455 mg (1.40 mmol) of cesium carbonate, 8.4 mg (0.037 mmol) of palladium(II) acetate and 18.6 mg (0.047 mmol) of racemic 2-(di-tert-butylphosphino)-1,1′-binaphthyl, evacuated and filled again with argon, 4 ml of dried toluene and 120 mg (0.93 mmol) of 2-chlorophenol are added, and the mixture is heated under argon and stirred under reflux overnight. For workup and purification, the mixture is filtered through Celite, the filtrate is concentrated, and the residue is taken up in methanol and separated by preparative HPLC (method 13). This affords 130 mg (60% of theory) of the target compound.

¹H NMR (400 MHz, DMSO-d₆): δ=1.57 (s, 9H), 7.33 (ddd, 1H), 7.37-7.47 (m, 2H), 7.63 (dd, 1H), 7.72 (br. t, 1H), 7.83 (br. d, 1H), 7.94 (br. s, 1H), 8.05 (br. d, 1H), 8.33 (d, 1H).

LC-MS (method 1): Rₜ=3.49 min; m/z=468 [M+H]⁺.

### Example 59A

### tert-Butyl 2-chloro-5-fluoro-6-(4-trifluoromethylphenyl)nicotinic acid

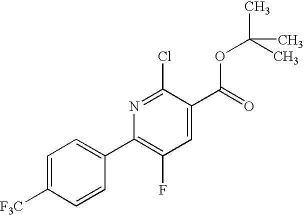

The title compound is prepared and purified analogously to Example 57A. Starting from 150 mg (0.56 mmol) of tert-butyl 2,6-dichloro-5-fluoronicotinate from Example 56A, 201 mg (95% of theory) of the target compound are obtained in this way.

¹H NMR (400 MHz, DMSO-d₆): δ=1.59 (s, 9H), 7.95 (AA′ part of an AA′BB′ system, br, 2H), 8.15 (BB′ part of an AA′BB′ system, br, 2H), 8.38 (d, 1H).

LC-MS (method 5): Rₜ=4.64 min; m/z=376 [M+H]⁺.

### Example 60A

### tert-Butyl 2-(2-chlorophenoxy)-5-fluoro-6-(4-trifluoromethylphenyl)nicotinate

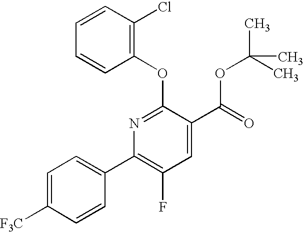

The title compound is prepared and purified analogously to Example 58A. Starting from 195 mg (0.42 mmol) of tert-butyl 2-chloro-5-fluoro-6-(4-trifluoromethylphenyl)nicotinate from Example 59A, 142 mg (73% of theory) of the target compound were thus obtained.

¹H NMR (400 MHz, DMSO-d₆): δ=1.56 (s, 9H), 7.31 (td, 1H), 7.37 (dd, 1H), 7.43 (ddd, 1H), 7.62 (dd, 1H), 7.84 (AA′ part of an AA′BB′ system, br, 2H), 7.89 (BB′ part of an AA′BB′ system, br, 2H), 8.35 (d, 1H).

LC-MS (method 11): Rₜ=3.26 min; m/z=468 [M+H]⁺.

### Example 61A

### Methyl 2,6-dichloro-4-methylnicotinate

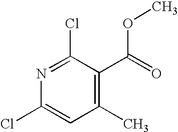

A solution of 10.3 g (45.9 mmol) of 2,6-dichloro-4-methylnicotinyl chloride [for preparation see DE 23 63 470-A1] in 20 ml of dichloromethane is added rapidly with stirring and cooling in a water/ice bath to 4.5 ml of pyridine in 100 ml of methanol. The mixture is stirred for a further 20 minutes and then concentrated under reduced pressure. The residue is taken up in ethyl acetate and washed successively with saturated aqueous sodium hydrogencarbonate solution, water and saturated aqueous sodium chloride solution. After drying over magnesium sulfate and filtration, the mixture is concentrated under reduced pressure. For purification, the mixture is filtered through 150 ml of silica gel in cyclohexane/ethyl acetate (1:1) and the eluent, after concentration, is crystallized from ethyl acetate/cyclohexane. After filtration and drying under reduced pressure, 5.8 g (58% of theory) of the target compound are obtained. A further 2.4 g (24% of theory) of the product are obtained from the mother liquor by another crystallization.

¹H NMR (400 MHz, DMSO-d₆): δ=2.33 (s, 3H), 3.93 (s, 3H) 7.66 (s, 1H).

LC-MS (method 1): Rₜ=2.30 min; m/z=220 [M+H]⁺.

### Example 62A

### Methyl 2-chloro-6-(2-fluoro-3-methoxyphenyl)-4-methylnicotinate

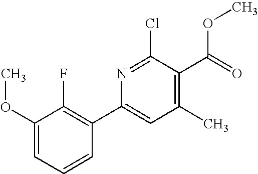

The title compound is prepared analogously to Example 57A. For purification, the crude product is separated first by preparative HPLC (method 9) and then by chromatography on silica gel with cyclohexane/ethyl acetate (10:1) as the eluent. Starting from 200 mg (0.91 mmol) of methyl 2,6-dichloro-4-methylnicotinate from Example 61A, 160 mg (57% of theory) of the target compound are thus obtained.

¹H NMR (400 MHz, DMSO-d₆): δ=2.39 (s, 3H), 3.90 (s, 3H), 3.95 (s, 3H), 7.25-7.35 (m, 2H), 7.38 (ddd, 1H), 7.79 (s, 1H).

LC-MS (method 1): Rₜ=2.70 min; m/z=310 [M+H]⁺.

### Example 63A

### Methyl 2-(2-chlorophenoxy)-6-(2-fluoro-3-methoxyphenyl)-4-methylnicotinate

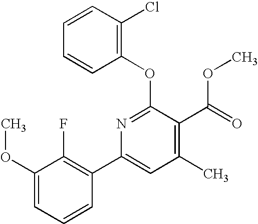

The title compound is prepared and purified analogously to Example 58A. After stirring overnight, in this case, to increase the reaction conversion, another 0.08 eq. of palladium acetate, 0.1 eq. of racemic 2-(di-tert-butylphosphino)-1,1′-binaphthyl and 250 mg of 4 Å molecular sieve are added, and the reaction mixture is heated to reflux with stirring over a further two nights. Starting from 74 mg (0.24 mmol) of methyl 2-chloro-6-(2-fluoro-3-methoxyphenyl)-4-methylnicotinate from Example 62A, 44 mg (46% of theory) of the target compound are thus obtained.

¹H NMR (400 MHz, DMSO-d₆): δ=2.41 (s, 3H), 3.84 (s, 3H), 3.92 (s, 3H), 7.01 (ddd, 1H), 7.12 (br. t, 1H), 7.20 (td, 1H), 7.30 (td, 1H), 7.36 (dd, 1H), 7.41 (ddd, 1H), 7.50 (d, 1H), 7.59 (dd, 1H).

LC-MS (method 3): Rₜ=4.23 min; m/z=402 [M+H]⁺.

### Example 64A

### 2-Chloro-6-(2,3-difluorophenyl)-4-trifluoromethylnicotinamide

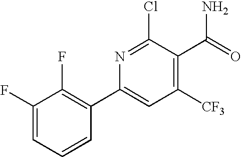

The title compound is prepared and purified analogously to Example 21A. Starting from 520 mg (2.00 mmol) of 2,6-dichloro-4-(trifluoromethyl)nicotinamide, 153 mg (23% of theory) of the target compound are thus obtained. Another preparative HPLC purification of mixed fractions from the first separation affords a further 95 mg (14% of theory) of the product.

¹H NMR (500 MHz, DMSO-d₆): δ=7.42 (td, 1H), 7.66 (q, 1H), 7.74 (t, 1H), 8.16 (s, 1H), 8.20 (s, 1H), 8.30 (s, 1H).

LC-MS (method 5): Rₜ=3.04 min; m/z=337 [M+H]⁺.

### Example 65A

### 2-(2-Chlorophenoxy)-6-(2,3-difluorophenyl)-4-trifluoromethylnicotinamide

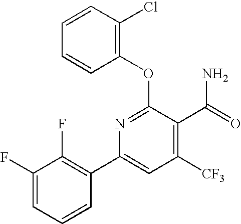

The title compound is prepared analogously to Example 16A. A portion of the product is obtained by precipitation from acetonitrile/water, a further fraction by preparative HPLC of the mother liquor according to method 8. Proceeding from 150 mg (0.45 mmol) of 2-chloro-6-(2,3-difluorophenyl)-4-trifluoromethylnicotinamide from Example 64A, 109 mg (57% of theory) of the target compound are thus obtained.

¹H NMR (400 MHz, DMSO-d₆): δ=7.22-7.38 (m, 3H), 7.38-7.49 (m, 2H), 7.54 (q, 1H), 7.63 (d, 1H), 7.88 (s, 1H), 8.02 (s, 1H), 8.25 (s, 1H).

LC-MS (method 5): Rₜ=3.57 min; m/z=429 [M+H]⁺.

### Example 66A

### 2-Chloro-6-(3,5-difluorophenyl)-4-trifluoromethylnicotinamide

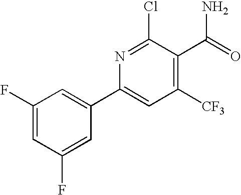

The title compound is prepared and purified analogously to Example 21A. On concentration of the corresponding HPLC separation fractions, the product precipitates out and is obtained by filtration and drying. Starting from 520 mg (2.00 mmol) of 2,6-dichloro-4-(trifluoromethyl)nicotinamide, 267 mg (40% of theory) of the target compound are thus obtained.

¹H NMR (400 MHz, DMSO-d₆): δ=7.48 (tt, 1H), 7.96 (m_{z}, 2H), 8.12 (s, 1H), 8.26 (s, 1H), 8.51 (s, 1H).

LC-MS (method 3): Rₜ=3.37 min; m/z=337 [M+H]⁺.

### Example 67A

### 2-(2-Chlorophenoxy)-6-(3,5-difluorophenyl)-4-trifluoromethylnicotinamide

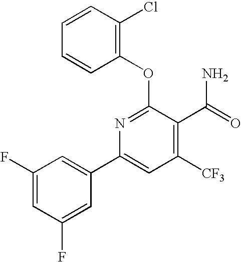

95 mg (0.74 mmol) of 2-chlorophenol and 308 mg (2.23 mmol) of potassium carbonate are added to 250 mg (0.74 mmol) of 2-chloro-6-(3,5-difluorophenyl)-4-trifluoromethylnicotinamide from Example 66A in 6 ml of DMF, and the reaction mixture is stirred at 60° C. overnight. For workup, the solid is filtered off, the mother liquor is concentrated under reduced pressure and the residue is taken up in water/ethyl acetate. The organic phase is removed, washed once more with water, dried over magnesium sulfate, filtered and concentrated, and the residue is dried under reduced pressure. This affords 211 mg (66% of theory) of the target compound.

¹H NMR (400 MHz, DMSO-d₆): δ=7.35 (tt, 1H), 7.37-7.46 (m, 2H), 7.50 (ddd, 1H), 7.59 (my, 2H), 7.68 (dd, 1H), 8.00 (br. s, 1H), 8.21 (s, 1H), 8.22 (br. s, 1H).

LC-MS (method 3): Rₜ=3.83 min; m/z=429 [M+H]⁺.

### Example 68A

### tert-Butyl 2-chloro-6-(2-fluoro-3-methoxyphenyl)-4-trifluoromethylnicotinate

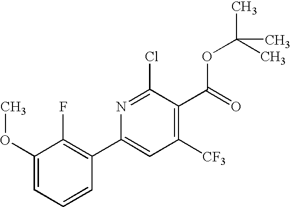

The title compound was prepared and purified analogously to Example 6A. Starting from 100 mg (0.32 mmol) of tert-butyl 2,6-dichloro-4-(trifluoromethyl)nicotinate from Example 34A, 82 mg (64% of theory) of the target compound are thus obtained.

¹H NMR (400 MHz, DMSO-d₆): δ=1.58 (s, 9H), 3.91 (s, 3H), 7.32 (t, 1H), 7.39 (td, 1H), 7.45 (ddd, 1H), 8.19 (s, 1H).

LC-MS (method 1): Rₜ=3.32 min; m/z=406 [M+H]⁺.

### Example 69A

### tert-Butyl 2-(2-chlorophenoxy)-6-(2-fluoro-3-methoxyphenyl)-4-trifluoromethylnicotinate

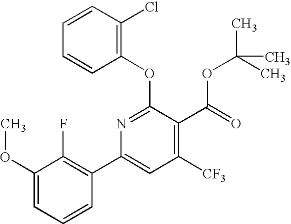

37 mg (0.29 mmol) of 2-chlorophenol and 80 mg (0.58 mmol) of potassium carbonate are added to a solution of 78 mg (0.19 mmol) of tert-butyl 2-chloro-6-(2-fluoro-3-methoxyphenyl)-4-trifluoromethylnicotinate from Example 68A in 3 ml of DMF. Subsequently, the mixture is stirred at 120° C. overnight. After filtration from the solid, the purification of the filtrate by means of preparative HPLC (method 13) gives 68 mg (71% of theory) of the target compound.

¹H NMR (400 MHz, DMSO-d₆): δ=1.56 (s, 9H), 3.86 (s, 3H), 7.06 (ddd, 1H), 7.18 (t, 1H), 7.27 (td, 1H), 7.35 (ddd, 1H), 7.40-7.51 (m, 2H), 7.64 (dd, 1H), 7.87 (s, 1H).

LC-MS (method 5): Rₜ=4.72 min; m/z=498 [M+H]⁺.

### Example 70A

### tert-Butyl 2-chloro-6-(3-fluoro-4-methylphenyl)nicotinate

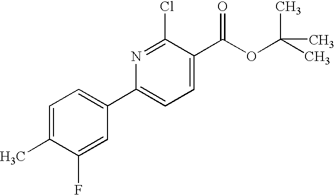

5.10 g (19.7 mmol) of tert-butyl 2,6-dichloronicotinate from Example 30A are initially charged in 106 ml of dioxane and degassed. 3.04 g (19.7 mmol) of (3-fluoro-4-methylphenyl)boronic acid and 59.2 ml (118.4 mmol) of a 2 M aqueous potassium carbonate solution are added and the mixture is stirred at RT for 10 min. Subsequently, 1.385 g (1.97 mmol) of bis(triphenylphosphine)palladium(II) chloride and 0.601 g (1.97 mmol) of tri-2-tolylphosphine are added and the reaction mixture is stirred at 60° C. overnight. After cooling, the reaction mixture is filtered through kieselguhr and the filtrate is concentrated to dryness under reduced pressure. The residue is admixed with ethyl acetate/water (1:1), the aqueous phase is removed and the organic phase is washed with water and with saturated sodium chloride solution. After drying over sodium sulfate, the solvent is removed under reduced pressure. The residue is chromatographed on silica gel (eluent: 85:15 cyclohexane/ethyl acetate). This affords 5.17 g (77% of theory) of the target compound.

¹H NMR (400 MHz, DMSO-d₆): δ=1.57 (s, 9H), 2.31 (s, 3H), 4.46 (t, 1H), 7.86-7.90 (m, 2H), 8.11 (d, 1H), 8.25 (d, 1H).

LC-MS (method 1): Rₜ=3.32 min; m/z=323 [M+H]⁺.

### Example 71A

### tert-Butyl 2-(4-bromo-2-fluorophenoxy)-6-(3-fluoro-4-methylphenyl)nicotinate

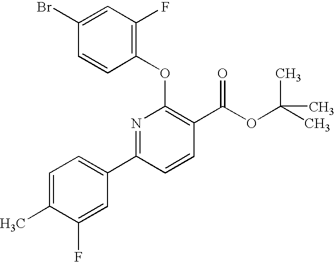

A mixture of 100 mg (0.31 mmol) of tert-butyl 2-chloro-6-(3-fluoro-4-methylphenyl)nicotinate from Example 70A, 60 mg (0.31 mmol) of 4-bromo-2-fluorophenol and 86 mg (0.62 mmol) of potassium carbonate in 1.8 ml of DMF is stirred at 100° C. for 24 h. After cooling, the reaction mixture is purified directly by preparative HPLC without further workup (eluent: acetonitrile/water with 0.1% formic acid, gradient 10:90→90:10). 29 mg (29% of theory) of the target compound are thus obtained.

LC-MS (method 14): Rₜ=1.81 min; m/z=476 [M+H]⁺.

### Working Examples

### Example 1

### 2-(2-Chlorophenoxy)-6-[4-(trifluoromethyl)phenyl]nicotinic acid

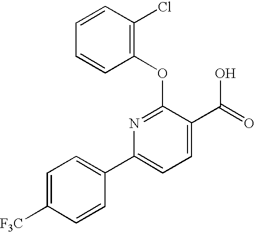

122 mg (1.35 mmol) of sodium chlorite, dissolved in 0.5 ml of water, and 131 mg (1.35 mmol) of amidosulfonic acid, likewise in 0.5 ml of water, are simultaneously added dropwise at 0° C. to 170 mg (0.45 mmol) of 2-(2-chlorophenoxy)-6-[4-(trifluoromethyl)phenyl]nicotinaldehyde (Example 11A) in 7.5 ml of THF. After stirring at 0° C. for 15 minutes, the reaction mixture is diluted with 20 ml of water and extracted twice with 20 ml each time of ethyl acetate. The combined organic phases are washed once with 50 ml of saturated aqueous sodium chloride solution and then concentrated under reduced pressure. The crude product thus obtained, after being taken up in methanol, is purified by preparative HPLC (method 10). This affords 166 mg (94% of theory) of the target compound.

¹H NMR (400 MHz, DMSO-d₆): δ=7.35 (td, 1H), 7.40 (dd, 1H), 7.46 (td, 1H), 7.64 (dd, 1H), 7.79 (d, 2H), 7.93 (d, 1H), 7.97 (d, 2H), 8.43 (d, 1H), 13.35 (br. s, 1H).

LC-MS (method 2): Rₜ=2.63 min; m/z=394 [M+H]⁺.

### Example 2

### 2-(2-Chlorophenoxy)-6-[3-(trifluoromethyl)phenyl]nicotinic acid

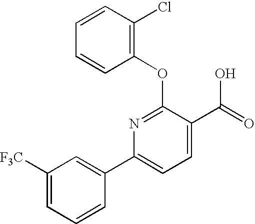

The title compound is prepared and purified analogously to Example 1. Starting from 130 mg (0.34 mmol) of 2-(2-chlorophenoxy)6-[3-(trifluoromethyl)phenyl]nicotinaldehyde from Example 12A, 126 mg (93% of theory) of the target compound are thus obtained.

¹H NMR (400 MHz, DMSO-d₆): δ=7.36 (td, 1H), 7.41 (dd, 1H), 7.47 (td, 1H), 7.64 (dd, 1H), 7.67 (d, 1H), 7.77 (d, 1H), 7.97 (d, 1H), 8.04 (br. s, 1H), 8.15 (d, 1H), 8.42 (d, 1H), 13.37 (br. s, 1H).

LC-MS (method 2): Rₜ=2.58 min; m/z=394 [M+H]⁺.

### Example 3

### 2-(2-Chlorophenoxy)-6-[4-chloro-3-(trifluoromethyl)phenyl]nicotinic acid

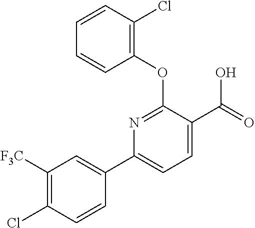

The title compound is prepared and purified analogously to Example 1. Starting from 140 mg (0.34 mmol) of 2-(2-chlorophenoxy)-6-[4-chloro-3-(trifluoromethyl)phenyl]nicotinaldehyde from Example 13A, 139 mg (96% of theory) of the target compound are thus obtained.

¹H NMR (400 MHz, DMSO-d₆): δ=7.36 (td, 1H), 7.41 (dd, 1H), 7.46 (td, 1H), 7.64 (dd, 1H), 7.81 (d, 1H), 7.98 (d, 1H), 8.11 (d, 1H), 8.17 (dd, 1H), 8.43 (d, 1H), 13.37 (br. s, 1H).

LC-MS (method 1): Rₜ=3.04 min; m/z=428 [M+H]⁺.

### Example 4

### 2-(2-Chlorophenoxy)-6-(4-fluoro-3-methylphenyl)nicotinic acid

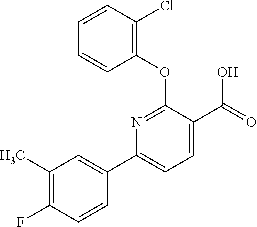

The title compound is prepared and purified analogously to Example 1. Starting from 110 mg (0.32 mmol) of 2-(2-chlorophenoxy)-6-(4-fluoro-3-methylphenyl)nicotinaldehyde from Example 14A, 111 mg (96% of theory) of the target compound are thus obtained.

¹H NMR (400 MHz, DMSO-d₆): δ=2.20 (s, 3H), 7.16 (t, 1H), 7.31-7.40 (m, 2H), 7.46 (ddd, 1H), 7.58-7.66 (m, 2H), 7.74 (dd, 1H), 7.79 (d, 1H), 8.36 (d, 1H), 13.21 (br. s, 1H).

LC-MS (method 1): Rₜ=2.80 min; m/z=358 [M+H]⁺.

### Example 5

### 2-(2-Chlorophenoxy)-6-(3-fluoro-4-methylphenyl)nicotinic acid

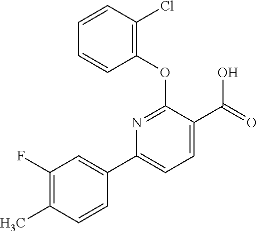

The title compound is prepared and purified analogously to Example 1. For further purification, it is chromatographed on silica gel (eluent: 20:1 dichlormethane/methanol). Starting from 100 mg (0.29 mmol) of 2-(2-chlorophenoxy)-6-(3-fluoro-4-methylphenyl)nicotinaldehyde from Example 15A, 63 mg (60% of theory) of the target compound are thus obtained.

¹H NMR (400 MHz, DMSO-d₆): δ=2.23 (s, 3H), 7.32 (t, 1H), 7.31-7.41 (m, 2H), 7.42-7.50 (m, 2H), 7.56 (dd, 1H), 7.64 (dd, 1H), 7.83 (d, 1H), 8.36 (d, 1H), 13.26 (br. s, 1H).

LC-MS (method 2): Rₜ=2.54 min; m/z=358 [M+H]⁺.

### Example 6

### 2-(2-Chlorophenoxy)-6-(2,3-difluorophenyl)nicotinic acid

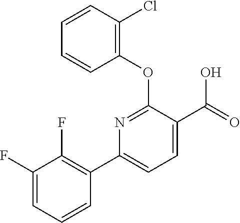

The title compound is prepared and purified analogously to Example 1. Starting from 105 mg (0.30 mmol) of 2-(2-chlorophenoxy)-6-(2,3-difluorophenyl)nicotinaldehyde from Example 16A, 100 mg (91% of theory) of the target compound are thus obtained.

¹H NMR (400 MHz, DMSO-d₆): δ=7.18-7.26 (m, 1H), 7.26-7.35 (m, 2H), 7.38 (dd, 1H), 7.40-7.54 (m, 2H), 7.61 (dd, 1H), 7.69 (dd, 1H), 8.43 (d, 1H), 13.39 (br. s, 1H).

LC-MS (method 2): Rₜ=2.38 min; m/z=362 [M+H]⁺.

### Example 7

### 2-(2-Chlorophenoxy)-6-(2-chlorophenyl)nicotinic acid

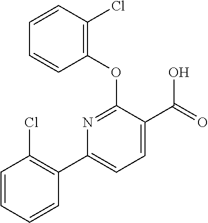

The title compound is prepared and purified analogously to Example 1. Starting from 79 mg (0.23 mmol) of 2-(2-chlorophenoxy)-6-(2-chlorophenyl)nicotinaldehyde from Example 17A, 66 mg (80% of theory) of the target compound are thus obtained.

¹H NMR (400 MHz, DMSO-d₆): δ=7.26 (ddd, 1H), 7.32-7.45 (m, 5H), 7.51 (dt, 1H), 7.53 (d, 1H), 7.56 (dd, 1H), 8.39 (d, 1H), 13.37 (br. s, 1H).

LC-MS (method 2): Rₜ=2.36 min; m/z=360 [M+H]⁺.

### Example 8

### 2-(2-Chlorophenoxy)-6-(2,3-dimethylphenyl)nicotinic acid

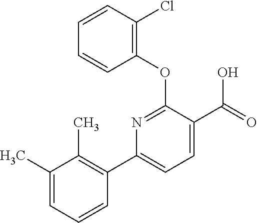

The title compound is prepared and purified analogously to Example 1. Starting from 150 mg (0.44 mmol) of 2-(2-chlorophenoxy)-6-(2,3-dimethylphenyl)nicotinaldehyde from Example 18A, 104 mg (66% of theory) of the target compound are thus obtained.

¹H NMR (400 MHz, DMSO-d₆): δ=1.94 (s, 3H), 2.20 (s, 3H), 7.08-7.15 (m, 2H), 7.15-7.22 (m, 1H), 7.26 (ddd, 1H), 7.30-7.35 (m, 2H), 7.39 (ddd, 1H), 7.56 (dd, 1H), 8.36 (d, 1H), 13.26 (br. s, 1H).

LC-MS (method 2): Rₜ=2.49 min; m/z=354 [M+H]⁺.

### Example 9

### 2-(2-Chlorophenoxy)-6-[3-(trifluoromethoxy)phenyl]nicotinic acid

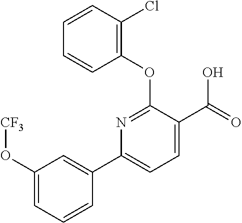

The title compound is prepared and purified analogously to Example 1. Starting from 130 mg (0.44 mmol) of 2-(2-chlorophenoxy)6-[3-(trifluoromethoxy)phenyl]nicotinaldehyde from Example 19A, 129 mg (95% of theory) of the target compound are thus obtained.

¹H NMR (400 MHz, DMSO-d₆): δ=7.35 (td, 1H), 7.38-7.43 (m, 2H), 7.46 (ddd, 1H), 7.63 (dd, 1H), 7.66 (br. s, 1H), 7.90 (br. d, 1H), 7.92 (d, 1H), 8.41 (d, 1H), 13.35 (br. s, 1H).

LC-MS (method 5): Rₜ=3.85 min; m/z=410 [M+H]⁺.

### Example 10

### 2-(2-Chlorophenoxy)-6-(2-fluoro-3-methoxyphenyl)nicotinic acid

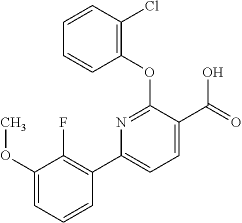

The title compound is prepared and purified analogously to Example 1. Starting from 90 mg (0.44 mmol) of 2-(2-chlorophenoxy)-6-(2-fluoro-3-methoxyphenyl)nicotinaldehyde from Example 20A, 90 mg (96% of theory) of the target compound are thus obtained.

¹H NMR (400 MHz, DMSO-d₆): δ=3.85 (s, 3H), 7.01 (ddd, 1H), 7.11 (t, 1H), 7.21 (td, 1H), 7.31 (td, 1H), 7.37 (dd, 1H), 7.43 (ddd, 1H), 7.61 (dd, 1H), 7.64 (dd, 1H), 8.40 (d, 1H), 13.34 (br. s, 1H).

LC-MS (method 5): Rₜ=3.44 min; m/z=374 [M+H]⁺.

### Example 11

### 2-(2-Chlorophenoxy)-6-(3-fluoro-4-methylphenyl)-4-(trifluoromethyl)nicotinic acid

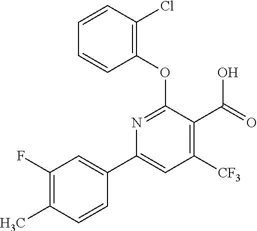

282 mg (4.10 mmol) of sodium nitrite are added in portions to 174 mg (0.41 mmol) of 2-(2-chlorophenoxy)-6-(3-fluoro-4-methylphenyl)-4-(trifluoromethyl)nicotinamide from Example 22A in a mixture of 2.0 ml of acetic acid and 6 ml of acetic anhydride, and the mixture is left to stir at RT overnight. 10 ml of water and 2 ml of concentrated hydrochloric acid are added and the mixture is stirred at RT for a further day. For workup, the mixture is concentrated and the residue is purified by preparative HPLC (method 8). This affords 22 mg (13% of theory) of the target compound.

¹H NMR (400 MHz, DMSO-d₆): δ=2.24 (s, 3H), 7.31-7.42 (m, 2H), 7.43-7.53 (m, 2H), 7.56-7.70 (m, 3H), 8.13 (s, 1H), 14.22 (br. s, 1H).

LC-MS (method 1): Rₜ=4.25 min; m/z=426 [M+H]⁺.

### Example 12

### 2-(2-Chloro-5-methoxyphenoxy)-6-(3-fluoro-4-methylphenyl)nicotinic acid

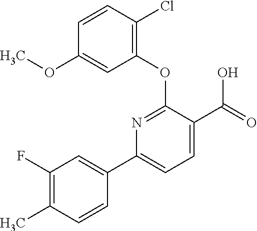

168 μl (0.168 mmol) of a 1 M aqueous lithium hydroxide solution and 2.0 ml of water are added to 45 mg (0.11 mmol) of methyl 2-(2-chloro-5-methoxyphenoxy)-6-(3-fluoro-4-methylphenyl)nicotinate from Example 24A in 0.5 ml of THF, and the mixture is stirred at RT overnight. For workup and purification, the mixture is acidified slightly with 1 N hydrochloric acid and separated by preparative HPLC (method 10). This affords 26 mg (60% of theory) of the target compound.

¹H NMR (400 MHz, DMSO-d₆): δ=2.23 (s, 3H), 3.78 (s, 3H), 6.92 (dd, 1H), 7.00 (d, 1H), 7.34 (t, 1H), 7.49 (dd, 1H), 7.52 (d, 1H), 7.58 (dd, 1H), 7.82 (d, 1H), 8.35 (d, 1H), 12.8-13.6 (broad, 1H).

LC-MS (method 3): Rₜ=3.93 min; m/z=388 [M+H]⁺.

### Example 13

### 2-(2-Chlorophenoxy)-6-phenylnicotinic acid

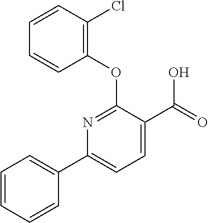

219 mg potassium hydroxide are added to 300 mg (0.98 mmol) of 2-(2-chlorophenoxy)-6-phenylnicotinonitrile from Example 25A in 20 ml of ethanol, and the mixture is heated to reflux with stirring for about 7 days. The mixture is concentrated, acidified with 1 N hydrochloric acid and admixed with water and ethyl acetate, the aqueous phase is extracted twice with ethyl acetate then with dichloromethane, and the combined organic phases are dried over sodium sulfate and finally concentrated. The purification is effected first by preparative HPLC, followed by chromatography on silica gel (removal of the secondary components first with an ethyl acetate/cyclohexane gradient, elution of the product with ethyl acetate and then ethanol). This affords 96 mg (30% of theory) of the target compound.

¹H NMR (400 MHz, DMSO-d₆): δ=7.26-7.34 (m, 2H), 7.35-7.46 (m, 4H), 7.58-7.64 (m, 1H), 7.70-7.79 (m, 3H), 8.24 (br. d, 1H), 12.5-13.5 (broad, 1H).

LC-MS (method 7): Rₜ=2.56 min; m/z=326 [M+H]⁺.

### Example 14

### 2-(2-Chlorophenoxy)-6-(4-fluorophenyl)nicotinic acid

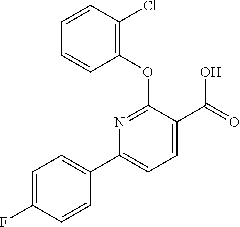

37 mg (0.11 mmol) of 2-(2-chlorophenoxy)-6-(4-fluorophenyl)nicotinonitrile from Example 26A are stirred in 2 ml of 70% aqueous sulfuric acid at 120° C. for 4 h. After cooling, the reaction mixture is added to ice-water and the precipitated solid is obtained by filtration, washing with water and drying under reduced pressure. The crude product thus obtained is purified by preparative HPLC (method 9). This affords 27 mg (69% of theory) of the target compound.

¹H NMR (400 MHz, DMSO-d₆): δ=7.24 (t, 2H), 7.28-7.40 (m, 2H), 7.45 (t, 1H), 7.63 (d, 1H), 7.73-7.89 (br. m, 3H), 8.34 (br. d, 1H), 12.5-14.0 (broad, 1H).

LC-MS (method 2): Rₜ=2.38 min; m/z=344 [M+H]⁺.

### Example 15

### 2-(2-Chlorophenoxy)-6-(4-chlorophenyl)nicotinic acid

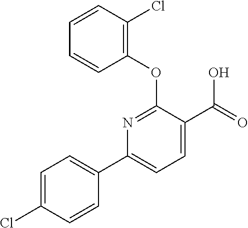

The title compound is prepared and purified analogously to Example 14. Starting from 310 mg (0.91 mmol) of 2-(2-chlorophenoxy)-6-(4-chlorophenyl)nicotinonitrile from Example 27A, 294 mg (90% of theory) of the target compound are thus obtained.

¹H NMR (400 MHz, DMSO-d₆): δ=7.31-7.41 (m, 2H), 7.42-7.52 (m, 3H), 7.63 (dd, 1H), 7.79 (d, 2H), 7.84 (d, 1H), 8.38 (d, 1H), 13.29 (s, 1H).

LC-MS (method 4): Rₜ=2.75 min; m/z=360 [M+H]⁺.

### Example 16

### 6′-Chloro-6-(2-chlorophenoxy)-2,3′-bipyridine-5-carboxylic acid

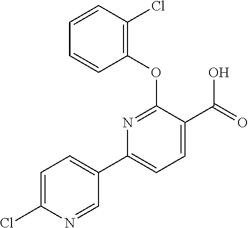

The title compound is prepared analogously to Example 1. The crude product is purified by preparative HPLC (method 10) three times. Starting from 135 mg (0.39 mmol) of 6′-chloro-6-(2-chlorophenoxy)-2,3′-bipyridine-5-carboxaldehyde from Example 29A, 62 mg (44% of theory) of the target compound are thus obtained.

¹H NMR (400 MHz, DMSO-d₆): δ=7.36 (ddd, 1H), 7.38-7.43 (m, 1H), 7.47 (ddd, 1H), 7.60 (d, 1H), 7.64 (dd, 1H), 7.94 (d, 1H), 8.16 (dd, 1H), 8.42 (d, 1H), 8.75 (d, 1H), 13.40 (br. s, 1H).

LC-MS (method 2): Rₜ=2.23 min; m/z=361 [M+H]⁺.

### Example 17

### 2-(2-Chloro-5-cyanophenoxy)-6-(3,5-difluorophenyl)nicotinic acid

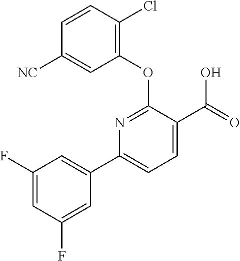

100.0 mg (0.307 mmol) of the compound from Example 33A are stirred in 1 ml of trifluoroacetic acid/dichloromethane (1:1) overnight. Thereafter, the mixture is taken up in 5 ml of water and precipitated crude product is isolated by filtration. Subsequently, the crude product is purified by means of preparative HPLC (eluent: acetonitrile/water with 0.1% formic acid, gradient 20:80→95:5). This affords 10 mg (12% of theory) of the target compound.

¹H NMR (400 MHz, DMSO-d₆): δ=7.35 (tt, 1H), 7.47 (m, 2H), 7.88 (dd, 1H), 7.92 (d, 1H), 8.00 (d, 1H), 8.08 (d, 1H), 8.45 (d, 1H), 13.47 (br. s, 1H).

LC-MS (method 11): Rₜ=2.27 min; m/z=387 [M+H]⁺.

### Example 18

### 2-(2,5-Difluorophenoxy)-6-(3,5-difluorophenyl)-4-(trifluoromethyl)nicotinic acid

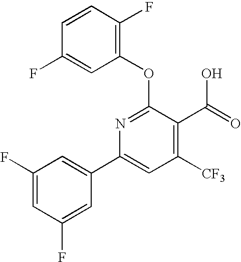

70.0 mg (0.144 mmol) of the compound from Example 36A are stirred in 1 ml of trifluoroacetic acid/dichloromethane (1:1) overnight. Thereafter, the mixture is taken up in 5 ml water and the precipitated crude product is isolated by filtration. Subsequently, the crude product is purified by means of preparative HPLC (eluent: acetonitrile/water with 0.1% formic acid, gradient 20:80→95:5). This affords 31 mg (50% of theory) of the target compound.

¹H NMR (400 MHz, DMSO-d₆): δ=7.29 (m_{z}, 1H), 7.40 (tt, 1H), 7.48-7.60 (m, 2H), 7.64 (m_{z}, 2H), 8.31 (s, 1H), 14.46 (br. s, 1H).

LC-MS (method 11): Rₜ=2.54 min; m/z=432 [M+H]⁺.

### Example 19

### 2-(4-Bromo-2-fluorophenoxy)-6-(3,5-difluorophenyl)-4-(trifluoromethyl)nicotinic acid

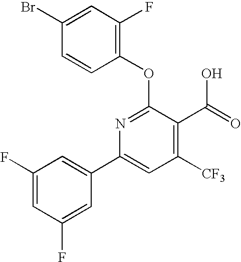

60.0 mg (0.109 mmol) of the compound from Example 37A are stirred in 0.8 ml of trifluoroacetic acid/dichloromethane (1:1) overnight. Thereafter, the mixture is taken up in 5 ml of water and the precipitated crude product is isolated by filtration. Subsequently, the crude product is purified by means of preparative HPLC (eluent: acetonitrile/water with 0.1% formic acid, gradient 20:80→95:5). This affords 31 mg (58% of theory) of the target compound.

¹H NMR (400 MHz, DMSO-d₆): δ=7.40 (tt, 1H), 7.47 (t, 1H), 7.56 (m_{z}, 1H), 7.63 (m_{z}, 2H), 7.87 (dd, 1H), 8.30 (s, 1H), 14.45 (br. s, 1H).

LC-MS (method 11): Rₜ=2.74 min; m/z=493 [M+H]⁺.

### Example 20

### 2-(2-Chloro-5-trifluoromethylphenoxy)-6-(2,3-difluorophenyl)nicotinic acid

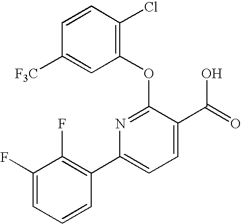

The title compound is prepared and purified analogously to Example 1. Starting from 68 mg (0.16 mmol) of 2-(2-chloro-5-trifluoromethylphenoxy)-6-(2,3-difluorophenyl)nicotinaldehyde from Example 38A, 69 mg (98% of theory) of the target compound are thus obtained.

¹H NMR (400 MHz, DMSO-d₆): δ=7.22 (tdd, 1H), 7.29 (ddt, 1H), 7.51 (dddd, 1H), 7.71 (dd, 1H), 7.73 (dd, 1H), 7.89 (d, 1H), 7.91 (d, 1H), 8.47 (d, 1H), 13.48 (br. s, 1H).

LC-MS (method 5): Rₜ=3.85 min; m/z=430 [M+H]⁺.

### Example 21

### 2-(2-Chloro-4-trifluoromethoxyphenoxy)-6-(2,3-difluorophenyl)nicotinic acid

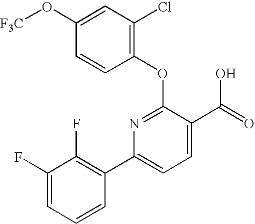

The title compound is prepared and purified analogously to Example 1. Starting from 57 mg (0.13 mmol) of 2-(2-chloro-4-trifluoromethoxyphenoxy)-6-(2,3-difluorophenyl)nicotinaldehyde from Example 39A, 57 mg (96% of theory) of the target compound are thus obtained.

¹H NMR (400 MHz, DMSO-d₆): δ=7.21 (tdd, 1H), 7.30 (ddt, 1H), 7.46-7.55 (m, 2H), 7.55 (d, 1H), 7.72 (dd, 1H), 7.80 (d, 1H), 8.46 (d, 1H), 13.47 (br. s, 1H).

LC-MS (method 5): Rₜ=3.95 min; m/z=446 [M+H]⁺.

### Example 22

### 2-(2-Chloro-4-methoxyphenoxy)-6-(2,3-difluorophenyl)nicotinic acid

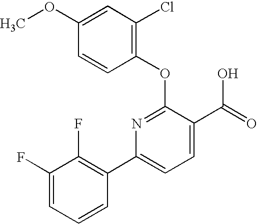

The title compound is prepared and purified analogously to Example 1. Starting from 36 mg (0.096 mmol) of 2-(2-chloro-4-methoxyphenoxy)-6-(2,3-difluorophenyl)nicotinaldehyde from Example 40A, 20 mg (53% of theory) of the target compound are thus obtained.

¹H NMR (400 MHz, DMSO-d₆): δ=3.77 (s, 3H), 6.90 (dd, 1H), 7.00 (d, 1H), 7.25 (tdd, 1H), 7.33 (ddt, 1H), 7.46-7.55 (m, 1H), 7.49 (d, 1H), 7.68 (dd, 1H), 8.43 (d, 1H), 13.40 (br. s, 1H).

LC-MS (method 1): Rₜ=2.73 min; m/z=392 [M+H]⁺.

### Example 23

### 2-(2-Fluoro-5-methylphenoxy)-6-(2,3-difluorophenyl)nicotinic acid

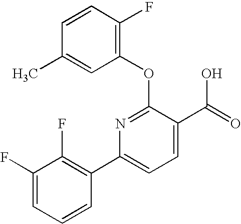

The title compound is prepared and purified analogously to Example 1. Starting from 31 mg (0.090 mmol) of 2-(2-fluoro-5-methylphenoxy)-6-(2,3-difluorophenyl)nicotinaldehyde from Example 41A, 31 mg (96% of theory) of the target compound are thus obtained.

¹H NMR (400 MHz, DMSO-d₆): δ=2.31 (s, 3H), 7.07-7.14 (m, 1H), 7.18 (dd, 1H), 7.21-7.30 (m, 2H), 7.34 (ddt, 1H), 7.51 (dddd, 1H), 7.69 (dd, 1H), 8.42 (d, 1H), 13.43 (br. s, 1H).

LC-MS (method 5): Rₜ=3.64 min; m/z=360 [M+H]⁺.

### Example 24

### 2-(2-Methoxyphenoxy)-6-(2,3-difluorophenyl)nicotinic acid

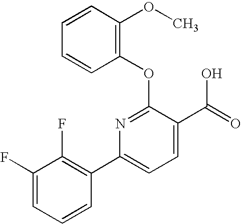

The title compound is prepared and purified analogously to Example 1. Starting from 21 mg (0.062 mmol) of 2-(2-methoxyphenoxy)-6-(2,3-difluorophenyl)nicotinaldehyde from Example 42A, 21 mg (96% of theory) of the target compound are thus obtained.

¹H NMR (400 MHz, DMSO-d₆): δ=3.68 (s, 3H), 7.00 (td, 1H), 7.13-7.31 (m, 5H), 7.48 (dddd, 1H), 7.61 (dd, 1H), 8.36 (d, 1H), 13.28 (br. s, 1H).

LC-MS (method 3): Rₜ=3.64 min; m/z=358 [M+H]⁺.

### Example 25

### 2-(2-Fluoro-5-trifluoromethylphenoxy)-6-(2,3-difluorophenyl)nicotinic acid

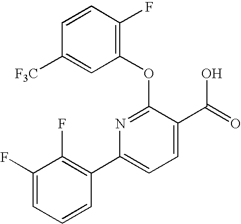

The title compound is prepared and purified analogously to Example 1. Starting from 67 mg (0.17 mmol) of 2-(2-fluoro-5-trifluoromethylphenoxy)-6-(2,3-difluorophenyl)nicotinaldehyde from Example 43A, 66 mg (95% of theory) of the target compound are thus obtained.

¹H NMR (400 MHz, DMSO-d₆): δ=7.23 (td, 1H), 7.32 (br. t, 1H), 7.51 (dddd, 1H), 7.67 (t, 1H), 7.71-7.79 (m, 2H), 7.92 (dd, 1H), 8.47 (d, 1H), 13.51 (br. s, 1H).

LC-MS (method 3): Rₜ=3.94 min; m/z=414 [M+H]⁺.

### Example 26

### 2-(2-Trifluoromethoxyphenoxy)-6-(2,3-difluorophenyl)nicotinic acid

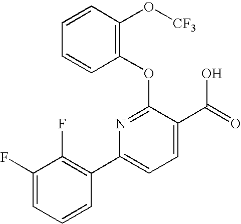

The title compound is prepared and purified analogously to Example 1. Starting from 70 mg (0.18 mmol) of 2-(2-trifluoromethoxyphenoxy)-6-(2,3-difluorophenyl)nicotinaldehyde from Example 44A, 69 mg (95% of theory) of the target compound are thus obtained.

¹H NMR (400 MHz, DMSO-d₆): δ=7.22 (td, 1H), 7.31 (t, 1H), 7.36-7.44 (m, 1H), 7.45-7.56 (m, 4H), 7.71 (dd, 1H), 8.43 (d, 1H), 13.40 (br. s, 1H).

LC-MS (method 3): Rₜ=3.90 min; m/z=412 [M+H]⁺.

### Example 27

### 2-(2-Fluorophenoxy)-6-(2,3-difluorophenyl)nicotinic acid

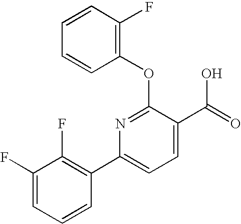

The title compound is prepared and purified analogously to Example 1. Starting from 32 mg (0.097 mmol) of 2-(2-fluorophenoxy)-6-(2,3-difluorophenyl)nicotinaldehyde from Example 45A, 31 mg (92% of theory) of the target compound are thus obtained.

¹H NMR (400 MHz, DMSO-d₆): δ=7.19-7.45 (m, 6H), 7.50 (dddd, 1H), 7.70 (dd, 1H), 8.43 (d, 1H), 13.45 (br. s, 1H).

LC-MS (method 3): Rₜ=3.67 min; m/z=346 [M+H]⁺.

### Example 28

### 2-(2-Chloro-5-methylphenoxy)-6-(2,3-difluorophenyl)nicotinic acid

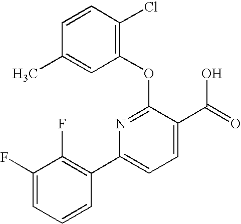

The title compound is prepared and purified analogously to Example 1. Starting from 26 mg (0.072 mmol) of 2-(2-chloro-5-methylphenoxy)-6-(2,3-difluorophenyl)nicotinaldehyde from Example 46A, 26 mg (96% of theory) of the target compound are thus obtained.

¹H NMR (400 MHz, DMSO-d₆): δ=2.32 (s, 3H), 7.12 (br. d, 1H), 7.18-7.27 (m, 2H), 7.31 (br. t, 1H), 7.44-7.55 (m, 1H), 7.47 (d, 1H), 7.67 (dd, 1H), 8.42 (d, 1H), 13.39 (br. s, 1H).

LC-MS (method 3): Rₜ=3.92 min; m/z=376 [M+H]⁺.

### Example 29

### 2-(2-Methylphenoxy)-6-(2,3-difluorophenyl)nicotinic acid

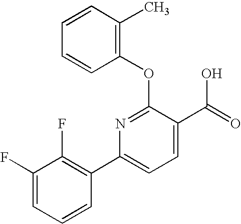

The title compound is prepared and purified analogously to Example 1. Starting from 27 mg (0.083 mmol) of 2-(2-methylphenoxy)-6-(2,3-difluorophenyl)nicotinaldehyde from Example 47A, 25 mg (88% of theory) of the target compound are thus obtained.

¹H NMR (400 MHz, DMSO-d₆): δ=2.12 (s, 3H), 7.11-7.29 (m, 4H), 7.29-7.36 (m, 2H), 7.49 (dddd, 1H), 7.64 (dd, 1H), 8.39 (d, 1H), 13.34 (br. s, 1H).

LC-MS (method 3): Rₜ=3.82 min; m/z=342 [M+H]⁺.

### Example 30

### 2-(5-Chloro-2-methylphenoxy)-6-(2,3-difluorophenyl)nicotinic acid

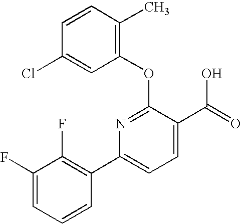

The title compound is prepared and purified analogously to Example 1. Starting from 19 mg (0.053 mmol) of 2-(5-chloro-2-methylphenoxy)-6-(2,3-difluorophenyl)nicotinaldehyde from Example 48A, 19 mg (96% of theory) of the target compound are thus obtained.

¹H NMR (400 MHz, DMSO-d₆): δ=2.11 (s, 3H), 7.21-7.31 (m, 3H), 7.35 (ddt, 1H), 7.37 (d, 1H), 7.51 (dddd, 1H), 7.68 (dd, 1H), 8.42 (d, 1H), 13.40 (br. s, 1H).

LC-MS (method 3): Rₜ=4.01 min; m/z=376 [M+H]⁺.

### Example 31

### 2-(2-Trifluoromethylphenoxy)-6-(2,3-difluorophenyl)nicotinic acid

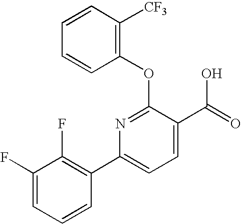

The title compound is prepared and purified analogously to Example 1. Starting from 61 mg (0.16 mmol) of 2-(2-trifluoromethylphenoxy)-6-(2,3-difluorophenyl)nicotinaldehyde from Example 49A, 62 mg (98% of theory) of the target compound are thus obtained.

¹H NMR (400 MHz, DMSO-d₆): δ=7.23 (dddd, 1H), 7.34 (ddt, 1H), 7.42-7.55 (m, 3H), 7.72 (dd, 1H), 7.75 (br. t, 1H), 7.82 (br. d, 1H), 8.44 (d, 1H), 13.40 (s, 1H).

LC-MS (method 3): Rₜ=3.85 min; m/z=396 [M+H]⁺.

### Example 32

### 2-(2,5-Difluorophenoxy)-6-(2,3-difluorophenyl)nicotinic acid

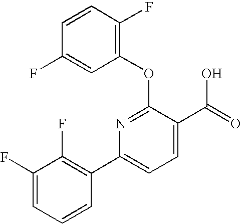

The title compound is prepared and purified analogously to Example 1. Starting from 55 mg (0.16 mmol) of 2-(2,5-difluorophenoxy)-6-(2,3-difluorophenyl)nicotinaldehyde from Example 50A, 56 mg (97% of theory) of the target compound are thus obtained.

¹H NMR (400 MHz, DMSO-d₆): δ=7.16-7.30 (m, 2H), 7.36 (ddt, 1H), 7.38-7.57 (m, 3H), 7.73 (dd, 1H), 8.45 (d, 1H), 13.49 (br. s, 1H).

LC-MS (method 3): Rₜ=3.72 min; m/z=364 [M+H]⁺.

### Example 33

### 2-(2-Chloro-5-methoxyphenoxy)-6-(2,3-difluorophenyl)nicotinic acid

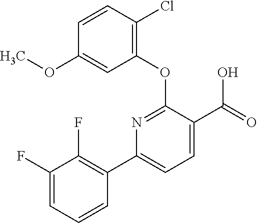

The title compound is prepared analogously to Example 1. Starting from 36 mg (0.38 mmol) of 2-(2-chloro-5-methoxyphenoxy)-6-(2,3-difluorophenyl)nicotinaldehyde from Example 51A, after purifying by preparative HPLC (method 10) twice, 24 mg (64% of theory) of the target compound are obtained.

¹H NMR (400 MHz, DMSO-d₆): δ=3.81 (s, 3H), 6.99 (dd, 1H), 7.19 (d, 1H), 7.24 (tdd, 1H), 7.28-7.36 (m, 1H), 7.31 (d, 1H), 7.50 (dddd, 1H), 7.66 (dd, 1H), 8.41 (d, 1H), 13.37 (br. s, 1H).

LC-MS (method 3): Rₜ=3.79 min; m/z=392 [M+H]⁺.

### Example 34

### 2-(2-Chloro-4-methoxyphenoxy)-6-(3-fluoro-4-methylphenyl)nicotinic acid

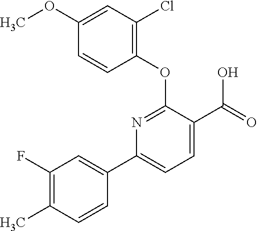

The title compound is prepared and purified analogously to Example 12. Starting from 60 mg (0.15 mmol) of methyl 2-(2-chloro-4-methoxyphenoxy)-6-(3-fluoro-4-methylphenyl)nicotinate from Example 52A, 56 mg (97% of theory) of the target compound are obtained.

¹H NMR (400 MHz, DMSO-d₆): δ=2.23 (s, 3H), 3.83 (s, 3H), 7.02 (dd, 1H), 7.22 (d, 1H), 7.31 (d, 1H), 7.34 (t, 1H), 7.49 (dd, 1H), 7.57 (dd, 1H), 7.80 (d, 1H), 8.34 (d, 1H), 12.8-13.7 (br, 1H).

LC-MS (method 3): Rₜ=3.94 min; m/z=388 [M+H]⁺.

### Example 35

### 2-(2,5-Difluorophenoxy)-6-(2-fluoro-3-methoxyphenyl)nicotinic acid

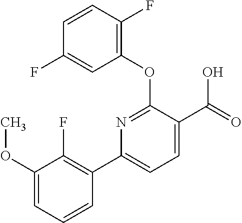

0.68 ml (8.86 mmol) of trifluoroacetic acid is added at 0° C. to 74 mg (0.17 mmol) of tert-butyl 2-(2,5-difluorophenoxy)-6-(2-fluoro-3-methoxyphenyl)nicotinate from Example 54A in 6.8 ml of dichloromethane, and the mixture is stirred at RT overnight. For workup and purification, the mixture is concentrated under reduced pressure, and the residue is taken up in a mixture of acetonitrile, water and a little DMF and separated by preparative HPLC (method 10). This affords 53 mg (82% of theory) of the target compound.

¹H NMR (400 MHz, DMSO-d₆): δ=3.86 (s, 3H), 7.06 (ddd, 1H), 7.11-7.28 (m, 3H), 7.40 (ddd, 1H), 7.46 (td, 1H), 7.68 (dd, 1H), 8.42 (d, 1H), 13.42 (br. s, 1H).

LC-MS (method 1): Rₜ=2.56 min; m/z=376 [M+H]⁺.

### Example 36

### 2-(2-Chlorophenoxy)-5-fluoro-6-(3-fluoro-4-methylphenyl)nicotinic acid

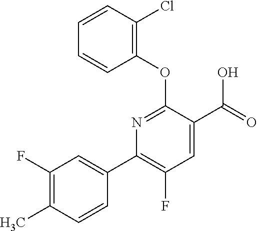

An argon-filled reaction flask is initially charged with 50 mg (0.17 mmol) of methyl 2-chloro-5-fluoro-6-(3-fluoro-4-methylphenyl)nicotinate from Example 55A, 164 mg (0.50 mmol) of cesium carbonate, 3.0 mg (0.013 mmol) of palladium(II) acetate and 6.7 mg (0.017 mmol) of racemic 2-(di-tert.-butylphosphino)-1,1′-binaphthyl, evacuated and filled again with argon, 3 ml of dried toluene and 43 mg (0.34 mmol) of 2-chlorophenol are added, and the mixture is heated under argon and stirred overnight under reflux. For workup and purification, the mixture is filtered through Celite, the filtrate is concentrated, and the residue is taken up in methanol and separated by preparative HPLC (method 9) three times. This affords 17 mg (27% of theory) of the target compound.

¹H NMR (400 MHz, DMSO-d₆): δ=2.24 (s, 3H), 7.30-7.41 (m, 4H), 7.41-7.50 (m, 2H), 7.64 (dd, 1H), 8.29 (d, 1H), 13.62 (br. s, 1H).

LC-MS (method 3): Rₜ=4.05 min; m/z=376 [M+H]⁺.

### Example 37

### 2-(2-Chlorophenoxy)-5-fluoro-6-(3-trifluoromethylphenyl)nicotinic acid

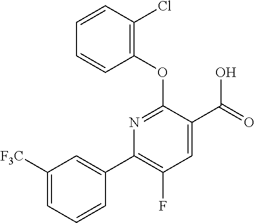

0.30 ml (3.9 mmol) of trifluoroacetic acid is added to 125 mg (0.27 mmol) of tert-butyl 2-(2-chlorophenoxy)-5-fluoro-6-(3-trifluoromethylphenyl)nicotinate from Example 58A in 3 ml of dichloromethane, and the mixture is stirred at RT overnight. For workup and purification, the mixture is concentrated under reduced pressure, taken up in acetonitrile and separated by preparative HPLC (method 10). This affords 99 mg (90% of theory) of the target compound.

¹H NMR (400 MHz, DMSO-d₆): δ=7.34 (ddd, 1H), 7.38-7.48 (m, 2H), 7.63 (dd, 1H), 7.72 (t, 1H), 7.83 (br. d, 1H), 7.92 (br. s, 1H), 8.04 (br. d, 1H), 8.35 (d, 1H), 13.72 (br. s, 1H).

LC-MS (method 11): Rₜ=2.55 min; m/z=412 [M+H]⁺.

### Example 38

### 2-(2-Chlorophenoxy)-5-fluoro-6-(4-trifluoromethylphenyl)nicotinic acid

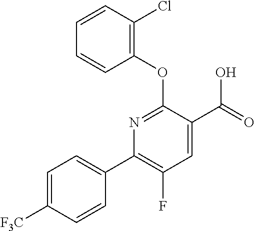

The title compound is prepared and purified analogously to Example 37. Starting from 135 mg (0.29 mmol) of tert-butyl 2-(2-chlorophenoxy)-5-fluoro-6-(4-trifluoromethylphenyl)nicotinate from Example 60A, 105 mg (88% of theory) of the target compound are thus obtained.

¹H NMR (400 MHz, DMSO-d₆): δ=7.32 (ddd, 1H), 7.36 (dd, 1H), 7.44 (ddd, 1H), 7.62 (dd, 1H), 7.83 (AA′ part of an AA′BB′ system, br, 2H), 7.86 (BB′ part of an AA′BB′ system, br, 2H), 8.36 (d, 1H), 13.73 (br. s, 1H).

LC-MS (method 11): Rₜ=2.58 min; m/z=412 [M+H]⁺.

### Example 39

### 2-(2-Chlorophenoxy)-6-(2-fluoro-3-methoxyphenyl)-4-methylnicotinic acid

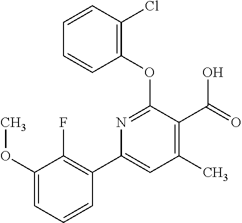

40 mg (0.10 mmol) of methyl 2-(2-chlorophenoxy)-6-(2-fluoro-3-methoxyphenyl)-4-methylnicotinate from Example 63A in 3 ml of THF are stirred with 3.6 mg (0.15 mmol) of lithium hydroxide and 0.3 ml of water first at RT for 4 h and then to reflux over two nights. For further completion of the conversion, the mixture is concentrated and taken up in dioxane, the same amount of lithium hydroxide and water is added and the mixture is heated under reflux for a further 5 h. For workup and purification, the mixture is acidified slightly with 1 N hydrochloric acid and separated directly by means of preparative HPLC (method 10). 33 mg (85% of theory) of the target compound are thus obtained.

¹H NMR (400 MHz, DMSO-d₆): δ=2.44 (s, 3H), 3.84 (s, 3H), 7.00 (ddd, 1H), 7.12 (br. t, 1H), 7.19 (td, 1H), 7.29 (td, 1H), 7.34 (dd, 1H), 7.41 (ddd, 1H), 7.47 (d, 1H), 7.59 (dd, 1H), 13.62 (br. s, 1H).

LC-MS (method 3): Rₜ=3.67 min; m/z=388 [M+H]⁺.

### Example 40

### 2-(2-Chlorophenoxy)-6-(2,3-difluorophenyl)-4-trifluoromethylnicotinic acid

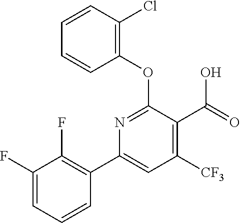

The title compound is prepared analogously to Example 11. The product is isolated by partial concentration of the reaction mixture and obtaining the precipitate formed by filtration. Starting from 110 mg (0.26 mmol) of 2-(2-chlorophenoxy)-6-(2,3-difluorophenyl)-4-trifluoromethylnicotinamide (Example 65A), 24 mg (22% of theory) of the target compound are obtained.

¹H NMR (400 MHz, DMSO-d₆): δ=7.27 (td, 1H), 7.31-7.39 (m, 2H), 7.40-7.50 (m, 2H), 7.54 (dddd, 1H), 7.64 (d, 1H), 7.92 (s, 1H), 14.0-14.8 (br, 1H).

LC-MS (method 3): Rₜ=4.01 min; m/z=430 [M+H]⁺.

### Example 41

### 2-(2-Chlorophenoxy)-6-(3,5-difluorophenyl)-4-trifluoromethylnicotinic acid

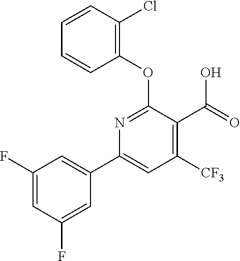

The title compound is prepared and purified analogously to Example 11. Starting from 180 mg (0.42 mmol) of 2-(2-chlorophenoxy)-6-(3,5-difluorophenyl)-4-trifluoromethylnicotinamide from Example 67A, 9.5 mg (5% of theory) of the target compound are obtained.

¹H NMR (400 MHz, DMSO-d₆): δ=7.33-7.44 (m, 2H), 7.45-7.54 (m, 2H), 7.59 (m_{z}, 2H), 7.69 (dd, 1H), 8.26 (s, 1H).

LC-MS (method 5): Rₜ=3.94 min; m/z=430 [M+H]⁺.

### Example 42

### 2-(2-Chlorophenoxy)-6-(2-fluoro-3-methoxyphenyl)-4-trifluoromethylnicotinic acid

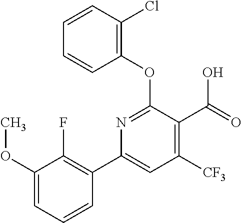

The title compound is prepared and purified analogously to Example 37. Starting from 63 mg (0.13 mmol) of tert-butyl 2-(2-chlorophenoxy)-6-(2-fluoro-3-methoxyphenyl)-4-trifluoromethylnicotinate from Example 69A, 50 mg (89% of theory) of the target compound are obtained.

¹H NMR (400 MHz, DMSO-d₆): δ=3.86 (s, 3H), 7.04 (ddd, 1H), 7.17 (t, 1H), 7.26 (td, 1H), 7.35 (ddd, 1H), 7.41-7.49 (m, 2H), 7.64 (dd, 1H), 7.86 (s, 1H), 14.36 (br. s, 1H).

LC-MS (method 3): Rₜ=3.81 min; m/z=442 [M+H]⁺.

### Example 43

### 2-(4-Bromo-2-fluorophenoxy)-6-(3-fluoro-4-methylphenyl)nicotinic acid

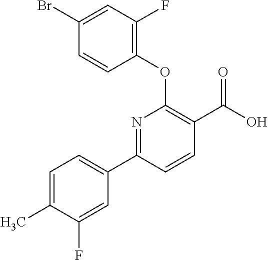

42 mg (0.09 mmol) of tert-butyl 2-(4-bromo-2-fluorophenoxy)-6-(3-fluoro-4-methylphenyl)-nicotinate from Example 71A and 34 mg (0.89 mmol) of sodium hydride (60% dispersion in mineral oil) are initially charged in 5 ml of THF. The reaction mixture is stirred at reflux temperature for 2 h. For workup, the solvent is removed under reduced pressure and the residue is adjusted to pH 1 with 1 N hydrochloric acid. After the volatile components have been removed on a rotary evaporator, the mixture is purified by means of preparative HPLC (eluent: acetonitrile/water, gradient 10:90→90:10). This affords 8 mg (22% of theory) of the target compound.

¹H NMR (400 MHz, DMSO-d₆): δ=2.24 (s, 3H), 7.34-7.40 (m, 2H), 7.48-7.60 (m, 3H), 7.80 (d, 1H), 7.86 (d, 1H), 8.36 (d, 1H), 13.34 (br. s, 1H).

LC-MS (method 1): Rₜ=2.80 min; m/z=421 [M+H]⁺.

### B. ASSESSMENT OF THE PHARMACOLOGICAL EFFICACY

The pharmacological action of the inventive compounds can be demonstrated in the following assays:

### 1. Cellular Transactivation Assay:

### a) Test Principle:

A cellular assay is used to identify activators of the peroxisome proliferator-activated receptor alpha (PPAR-alpha).

Since mammalian cells contain different endogenous nuclear receptors which can complicate unambiguous interpretation of the results, an established chimera system is used, in which the ligand binding domain of the human PPARα-receptor is fused to the DNA binding domain of the yeast transcription factors GAL4. The GAL4-PPARα chimera thus formed is co-transfected and expressed stably in CHO cells with a reporter construct.

### b) Cloning:

The GAL4-PPARα expression construct contains the ligand binding domain of PPARα (amino acids 167-468), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, leads to the expression of firefly luciferase (Photinus pyralis) after activation and binding of GAL4-PPARα.

### c) Test Procedure:

The day before the test, CHO (chinese hamster ovary) cells which stably express the above-described GAL4-PPARα chimera and luciferase reporter gene construct are plated out in 96-hole microtiter plates with 1×10³ cells in medium (Optimem, GIBCO), 2% activated carbon-purified fetal calf serum (Hyclone), 1.35 mM sodium pyruvate (GIBCO), 0.2% sodium bicarbonate (GIBCO), and kept in a cell incubator (air humidity 96%, 5% v/v CO₂, 37° C.). On the day of the test, the substances to be tested are taken up in abovementioned medium, but without addition of calf serum, and added to the cells. After a stimulation time of 6 h, the luciferase activity is measured with the aid of a video camera. The relative light units measured give a sigmoid stimulation curve as a function of the substance concentration. The EC₅₀ values are calculated with the aid of the computer program GraphPad PRISM (Version 3.02).

The table which follows lists the EC₅₀ values of representative example compounds:

TABLE

| Example No. | EC₅₀ [nM] |
|---|---|
| 4 | 157 |
| 5 | 33 |
| 11 | 14 |
| 16 | 870 |
| 17 | 69 |
| 26 | 76 |
| 36 | 34 |
| 38 | 360 |
| 42 | 541 |

### 2. Fibrinogen Determination:

To determine the action on the plasma fibrinogen concentration, male Wistar rats or NMRI mice are treated with the substance to be studied by gavage administration or by means of addition to feed for a period of 4-9 days. Under terminal anesthesia, citrate blood is then obtained by heart puncture. The plasma fibrinogen level is determined by the Claus method [A. Claus, Acta Haematol. 17, 237-46 (1957)] by measuring the thrombin time with human fibrinogen as the standard.

3. Test Description for the Discovery of Pharmacologically Active Substances which Increase Apoprotein A1 (ApoA1) and HDL Cholesterol (HDL-C) in the Serum of Transgenic Mice which have been Transfected with the Human ApoA1 Gene (hApoA1) or Lower the Serum Triglycerides (TG):

The substances which are to be examined in vivo for their HDL-C-increasing action are administered orally to male transgenic hApoA1 mice. One day before the start of the experiment, the animals are assigned randomly to groups with the same number of animals, generally n=7-10. Over the entire experiment, the animals have drinking water and feed ad libitum. The substances are administered orally every day for 7 days. For this purpose, the test substances are dissolved in a solution of Solutol HS 15+ethanol+sodium chloride solution (0.9%) in a ratio of 1+1+8 or in a solution of Solutol HS 15+sodium chloride solution (0.9%) in a ratio of 2+8. The dissolved substances are administered in a volume of 10 ml/kg of body weight with a gavage. The control group used is composed of animals which are treated in exactly the same way but receive only the solvent (10 ml/kg of body weight) without test substance.

Before the first substance administration, blood is taken from every mouse by puncturing the retroorbital venous plexus to determine ApoA1, serum cholesterol, HDL-C and serum triglycerides (TG) (zero value). Subsequently, the test substance is administered to the animals for the first time with a gavage. 24 hours after the last substance administration (on the 8th day after the start of treatment), blood is again taken from each animal by puncturing the retroorbital venous plexus to determine the same parameters. The blood samples are centrifuged and, after obtaining the serum, TG, cholesterol, HDL-C and human ApoA1 are determined with a Cobas Integra 400 plus unit (Cobas Integra, from Roche Diagnostics GmbH, Mannheim) using the particular cassettes (TRIGL, CHOL2, HDL-C and APOAT). HDL-C is determined by gel filtration and post-column derivatization with MEGA cholesterol reagent (from Merck KGaA) analogously to the method of Garber et al. [J. Lipid Res. 41, 1020-1026 (2000)].

The action of the test substances on the HDL-C, hApoA1 and TG concentrations is determined by subtracting the measurement from the 1st blood sample (zero value) from the measurement of the 2nd blood sample (after treatment). The differences of all HDL-C, hApoA1 and TG values of one group are averaged and compared to the mean of the differences of the control group. The statistical evaluation is effected with Student t's test after previously checking the variances for homogeneity.

Substances which increase the HDL-C of the animals treated, compared to the control group, in a statistically significant manner (p<0.05) by at least 20%, or lower the TG in a statistically significant manner (p<0.05) by at least 25%, are considered to be pharmacologically active.

### 4. DOCA/Salt Model:

The administration of deoxycorticosterone acetate (DOCA) in combination with a high-salt diet and removal of one kidney induces hypertension in rats, which is characterized by a relatively low renin level. A consequence of this endocrine hypertension (DOCA is a direct precursor of aldosterone), depending on the DOCA concentration selected, is hypertrophy of the heart and further end organ damage, for example to the kidney, which is characterized by features including proteinuria and glomerulosclerosis. In this rat model, it is thus possible to examine test substances for antihypertrophic and end organ-protective action present.

Male Sprague Dawley (SD) rats of about 8 weeks of age (body weight between 250 and 300 grams) are uninephrectomized on the left side. To this end, the rats are anesthetized with 1.5-2% isoflurane in a mixture of 66% N₂O and 33% O₂, and the kidney is removed through a flank section. The later control animals used are so-called sham-operated animals from which no kidney has been removed.

Uninephrectomized SD rats received 1% sodium chloride in drinking water and, once per week, a subcutaneous injection of desoxycorticosterone acetate (dissolved in sesame oil; from Sigma) injected between the shoulder blades (high dose: 100 mg/kg/week s.c.; normal dose: 30 mg/kg/week s.c.).

The substances which are to be examined in vivo for their protective action are administered by gavage or via the feed (from Ssniff) or drinking water. One day before the start of the experiment, the animals are randomized and assigned to groups with the same number of animals, generally n=10. Over the entire experiment, drinking water and feed are available to the animals ad libitum.

The substances are administered once per day for 4-6 weeks via gavage, feed or drinking water. The placebo group used is animals which have been treated in exactly the same way but receive either only the solvent or the feed or drinking water without test substance.

The action of the test substances is determined by measuring hemodynamic parameters [blood pressure, heart rate, intropy (dp/dt), relaxation time (tau), maximum left-ventricular pressure, left ventricular end-diastolic pressure (LVEDP)], weight determination of heart, kidney and lung, measure of protein excretion and by measuring the gene expression of biomarkers (e.g. ANP, atrial natriuretic peptide, and BNP, brain natriuretic peptide) by means of RT/TaqMan-PCR after RNA isolation from cardiac tissue.

The statistical evaluation is effected with Student t's test after previously checking the variances for homogeneity.

### C. WORKING EXAMPLES FOR PHARMACEUTICAL COMPOSITIONS

The inventive compounds can be converted to pharmaceutical formulations as follows:

### Tablet:

### Composition:

100 mg of the inventive compound, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm

### Production:

The mixture of inventive compounds, lactose and starch is granulated with a 5% solution (m/m) of the PVP in water. After drying, the granule is mixed with the magnesium stearate for 5 minutes. This mixture is pressed with a customary tablet press (see above for format of the tablet). The guide value used for the compression is a pressing force of 15 kN.

### Orally Administerable Suspension:

### Composition:

1000 mg of the inventive compound, 1000 mg of ethanol (96%), 400 mg of Rhodigel® (xanthan gum from FMC, Pennsylvania, USA) and 99 g of water.

10 ml of oral suspension corresponds to a single dose of 100 mg of the inventive compounds.

### Production:

The Rhodigel is suspended in ethanol, and the inventive compound is added to the suspension. The water is added with stirring. The mixture is stirred for approx 6 h until the swelling of the Rhodigel is complete.

### Orally Administerable Solution:

### Composition:

500 mg of the inventive compound, 2.5 g of polysorbate and 97 g of polyethylene glycol 400.20 g of oral solution corresponds to a single dose of 100 mg of the inventive compound.

### Production:

The inventive compound is suspended in the mixture of polyethylene glycol and polysorbate with stirring. The stirring operation is continued up to complete dissolution of the inventive compound.

i.v. Solution:

The inventive compound is dissolved in a physiologically compatible solvent (e.g. isotonic saline, 5% glucose solution and/or 30% PEG 400 solution) in a concentration below the saturation solubility. The solution is filtered under sterile conditions and filled into sterile and pyrogen-free injection vessels.

1. A compound of the formula (I) 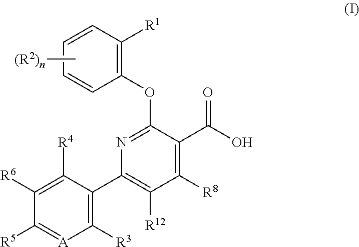
in which
R¹ is halogen, cyano, (C₁-C₄)-alkyl, trifluoromethyl, (C₁-C₄)-alkoxy or trifluoromethoxy,
R² is a substituent selected from the group of halogen, cyano, (C₁-C₆)-alkyl, (C₁-C₆)-alkoxy and —NR⁹—C(═O)—R¹⁰, in which alkyl and alkoxy may in turn be substituted by hydroxyl, (C₁-C₄)-alkoxy, amino, mono-(C₁-C₄)-alkylamino or di-(C₁-C₄)-alkylamino, or up to pentasubstituted by fluorine, and
R⁹ is hydrogen or (C₁-C₆)-alkyl
and
R¹⁰ is hydrogen, (C₁-C₆)-alkyl or (C₁-C₆)-alkoxy,
n is 0, 1, 2 or 3,
where, in the case that the substituent R² occurs more than once, its definitions may be identical or different,
A is N or C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, (C₁-C₄)-alkyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkyl-amino or di-(C₁-C₄)-alkylamino or up to pentasubstituted by fluorine,
R⁸ is hydrogen, methyl or trifluoromethyl
and
R¹² is hydrogen or fluorine,
and the salts, solvates and solvates of the salts thereof.

2. A compound of the formula (I) as claimed in claim 1, in which
R¹ is halogen, cyano or (C₁-C₄)-alkyl,
R² is a substituent selected from the group of halogen, cyano, (C₁-C₆)-alkyl, (C₁-C₆)-alkoxy and —NR⁹—C(═O)—R¹⁹, in which alkyl and alkoxy may in turn be substituted by hydroxyl, (C₁-C₄)-alkoxy, amino, mono-(C₁-C₄)-alkylamino or di-(C₁-C₄)-alkylamino, or up to pentasubstituted by fluorine, and
R⁹ is hydrogen or (C₁-C₆)-alkyl
and
R¹⁰ is hydrogen, (C₁-C₆)-alkyl or (C₁-C₆)-alkoxy,
n is 0, 1, 2 or 3,
where, in the case that the substituent R² occurs more than once, its definitions may be identical or different,
A is N or C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, (C₁-C₄)-alkyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkyl-amino or di-(C₁-C₄)-alkylamino or up to pentasubstituted by fluorine,
R⁸ is hydrogen, methyl or trifluoromethyl
and R¹² is hydrogen,
and the salts, solvates and solvates of the salts thereof.

3. A compound of the formula (I) as claimed in claim 1, in which
R¹ is halogen, cyano or (C₁-C₆)-alkyl,
R² is a substituent selected from the group of halogen, cyano, (C₁-C₆)-alkyl and (C₁-C₆)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to pentasubstituted by fluorine,
n is 0, 1 or 2,
where, in the case that the substituent R² occurs twice, its definitions may be the same or different,
A is C—R⁷,
R³ is hydrogen or fluorine,
R⁴ is hydrogen, fluorine, chlorine, cyano or (C₁-C₄)-alkyl,
R⁵ is hydrogen, halogen, nitro, cyano, amino, trifluoromethyl, (C₁-C₄)-alkyl, trifluoromethoxy or (C₁-C₄)-alkoxy,
R⁶ and R⁷ are the same or different and are each independently hydrogen, halogen, nitro, cyano, (C₁-C₆)-alkyl or (C₁-C₆)-alkoxy, in which alkyl and alkoxy may in turn be substituted by hydroxyl, (C₁-C₄)-alkoxy, amino, mono-(C₁-C₄)-alkyl-amino or di-(C₁-C₄)-alkylamino or up to pentasubstituted by fluorine,
R⁸ is hydrogen, methyl or trifluoromethyl
and
R¹² is fluorine,
and the salts, solvates and solvates of the salts thereof.

4. A compound of the formula (I) as claimed in claim 1 in which
R¹ is fluorine, chlorine, bromine, cyano or (C₁-C₄)-alkyl,
R² is a substituent selected from the group of fluorine, chlorine, bromine, cyano, (C₁-C₄)-alkyl and (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
n is 0, 1 or 2,
where, in the case that the substituent R² occurs twice its definitions may be the same or different,
A is N or C—R⁷,
R³ is hydrogen or fluorine,
R⁴ is hydrogen, fluorine, chlorine or methyl,
R⁵ is hydrogen, fluorine, chlorine, cyano, trifluoromethyl, trifluoromethoxy or (C₁-C₄)-alkoxy,
R⁶ and R¹ are the same or different and are each independently hydrogen, fluorine, chlorine, bromine, cyano, (C₁-C₄)-alkyl or (C₁-C₄)-alkoxy, in which alkyl and alkoxy may in turn be substituted by hydroxyl, (C₁-C₄)-alkoxy, amino, mono-(C₁-C₄)-alkylamino or di-(C₁-C₄)-alkylamino or up to trisubstituted by fluorine,
R⁸ is hydrogen, methyl or trifluoromethyl
and
R¹² is hydrogen,
and the salts, solvates and solvates of the salts thereof.

5. A compound of the formula (I) as claimed in claim 1 in which
R¹ is fluorine, chlorine, bromine, cyano or (C₁-C₄)-alkyl,
R² is a substituent selected from the group of fluorine, chlorine, bromine, cyano, (C₁-C₄)-alkyl, trifluoromethyl, (C₁-C₄)-alkoxy and trifluoromethoxy,
n is 0, 1 or 2,
where, in the case that the substituent R² occurs twice, its definitions may be the same or different,
A is C—R⁷,
R³ is hydrogen or fluorine,
R⁴ is hydrogen, fluorine, chlorine or methyl,
R⁵ is hydrogen, fluorine, chlorine, cyano, trifluoromethyl, (C₁-C₄)-alkyl, trifluoromethoxy or (C₁-C₄)-alkoxy,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
R⁸ is hydrogen, methyl or trifluoromethyl
and
R¹² is fluorine,
and the salts, solvates and solvates of the salts thereof.

6. A compound of the formula (I) as claimed in claim 1 in which
R¹ is fluorine, chlorine, bromine, cyano or methyl,
R² is a substituent selected from the group of fluorine, chlorine, bromine, cyano, (C₁-C₄)-alkyl, trifluoromethyl, (C₁-C₄)-alkoxy and trifluoromethoxy,
n is 0, 1 or 2,
where, in the case that the substituent R² occurs twice, its definitions may be the same or different,
A is C—R⁷,
R³ is hydrogen,
R⁴ is hydrogen or fluorine,
R⁵ is hydrogen, fluorine, chlorine, methyl or trifluoromethyl,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
R⁸ is hydrogen or trifluoromethyl
and
R¹² is hydrogen,
and the salts, solvates and solvates of the salts thereof.

7. A compound of the formula (I) as claimed in claim 1 in which
R¹ is fluorine, chlorine or cyano,
R² is a substituent selected from the group of fluorine, chlorine, (C₁-C₄)-alkoxy and trifluoromethoxy,
n is 0 or 1,
A is C—R⁷,
R³ and R⁴ are each hydrogen,
R⁵ is hydrogen, fluorine, chlorine, methyl or trifluoromethyl,
R⁶ and R⁷ are the same or different and are each independently hydrogen, fluorine, chlorine, bromine, cyano, (C₁-C₄)-alkyl, trifluoromethyl, (C₁-C₄)-alkoxy or trifluoromethoxy,
R⁸ is hydrogen
and
R¹² is fluorine,
and the salts, solvates and solvates of the salts thereof.

8. A process for preparing compounds of the formula (I) as defined in claim 1, wherein a compound of the formula (II) 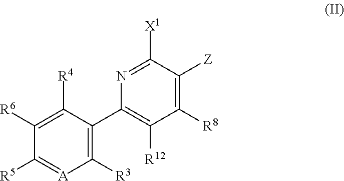
in which A, R³, R⁴, R⁵, R⁶, R⁸ and R¹² are each defined as specified in claim 1,
X¹ is a suitable leaving group, for example halogen,
and
Z is the —CHO, —CONH₂, —CN or —COOK¹¹ group in which
R¹¹ is (C₁-C₄)-alkyl,
in an inert solvent in the presence of a base, is reacted with a compound of the formula (III) 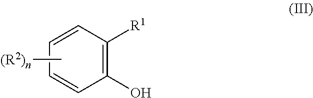
in which R¹, R² and n are each defined as specified in claim 1
to give compounds of the formula (IV) 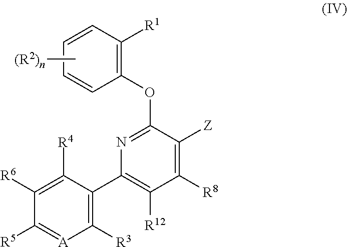
in which A, R¹, R², R³, R⁴, R⁵, R⁶, R⁸, R¹², Z and n are defined as specified above, and these compounds are converted to the carboxylic acids of the formula (I) by oxidation when Z is —CHO, or by basic or acidic hydrolysis when Z is —CN or —COOR¹¹, or by acidic or basic hydrolysis or by reaction with sodium nitrite and subsequent treatment with hydrochloric acid when Z is —CONH₂,
and the compounds of the formula (I) are optionally reacted with the corresponding (i) solvents and/or (ii) bases or acids to give their solvates, salts and/or solvates of the salts.

9. A compound of the formula (I) as defined in claim 1 for the treatment and/or prophylaxis of diseases.

10. (canceled)

11. A pharmaceutical composition comprising a compound of the formula (I) as defined in claim 1 in combination with an inert, non-toxic, pharmaceutically suitable assistant.

12. The pharmaceutical composition as claimed in claim 11 further comprising one or more active ingredients selected from the group consisting of HMG-CoA reductase inhibitors, diuretics, beta-receptor blockers, organic nitrates and NO donors, ACE inhibitors, angiotensin AII antagonists, aldosterone and mineralocorticoid receptor antagonists, vasopressin receptor antagonists, thrombocyte aggregation inhibitors and anticoagulants.

13. The pharmaceutical composition as claimed in claim 11 for the treatment and/or prophylaxis of dyslipidemias, arteriosclerosis and heart failure.

14. A method for the treatment and/or prophylaxis of dyslipidemias, arteriosclerosis and heart failure in humans and animals by administering an effective amount of at least one compound of the formula (I) as defined in claim 1.

15. A method for the treatment and/or prophylaxis of dyslipidemias, arteriosclerosis and heart failure in humans and animals by administering an effective amount of the pharmaceutical composition as claimed in claim 11.
